(19) United States
(12) Patent Application Publication
(10) Pub. No.: US 2006/0189639 A1
(43) Pub. Date: Aug. 24, 2006

(54) Antagonists of the mGlu receptor and uses thereof

(76) Inventors: Andrew O. Stewart, Libertyville, IL (US); Guo Zhu Zheng, Lake Bluff, IL (US); Teodozyj Kolasa, Lake Villa, IL (US); Pramila A. Bhatia, Libertyville, IL (US); Jerome F. Daanen, Racine, WI (US); Steven P. Latshaw, Round Lake Beach, IL (US); Xueqing Wang, Evanston, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/333,035

(22) Filed: Jan. 17, 2006

Related U.S. Application Data
(60) Provisional application No. 60/646,729, filed on Jan. 24, 2005.

(51) Int. Cl. A61K 31/519; C07D 498/14

(52) U.S. Cl. 514/267; 544/250

(57) ABSTRACT

The present invention discloses compounds of general formula (I) 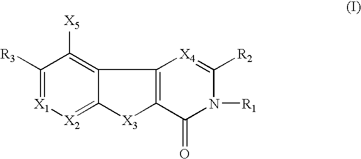
wherein X₁-X₄ and R₁-R₃ are as defined in the description. The present invention also discloses methods of treatment for pain, neurodegeneration and convulsive states in a host mammal in need thereof, and pharmaceutical compositions including those compounds.

This application claims priority to the provisional application Ser. No. 60/646,729 filed on Jan. 24, 2005.

### FIELD OF THE INVENTION

The present invention relates to compounds of formula (I) that are antagonists of the mGlu receptor and are useful for treating glutamate-induced diseases of the central nervous system, as well as formulations comprising such compounds.

### BACKGROUND OF THE INVENTION

Glutamate is one of the excitatory neurotransmitter in the central nervous system (CNS). Glutamate binds to both ligand-gated ion channels (ionotropic receptors) and G-protein coupled (metabotropic) receptors. Glutamate metabotropic receptors (mGluRs) are a subfamily of the G-protein-coupled receptors (GPCR) and comprise three (3) different groups, I, II and III—with eight distinct subtypes of mGluRs, namely mGluR1 to mGluR8—on the basis of primary sequence similarity, signal transduction linkages and pharmacological profile. Members of the mGluRs family have unique pharmacological properties and function to modulate the presynaptic release of glutamate and the post-synaptic sensitivity of the cell to glutamate excitation. Group I mGluRs is linked to stimulation of phospholipase C activity and includes mGluR1 and mGluR5; Group II mGluRs is linked to inhibition of adenylyl cyclase activity and includes mGluR2 and mGluR3; and Group III mGluRs is linked to inhibition of adenylyl cyclase activity and includes mGluR4, mGluR6, mGluR7 and mGluR8.

Elucidation of the physiological roles of particular mGluRs, and the mGluR-associated pathophysiological processes that affect the CNS have yet to be defined. Several pieces of evidence suggest an important involvement of mGluRs in pain sensation and analgesia (Meller et al., Neuroreport Vol. 4: 879 (1993). Knock out animals exhibit a reduction in excitatory responses to C-fiber (pain) inputs.

mGluR5 are located postsynaptically in neurons and glial cells enhancing glutamate and GABA neuronal transmission. Pharmacological studies with the non-competitive mGluR5 antagonist 2-methyl-6-(phenylethynil)pyridine (MPEP) also supports a role of these receptors in pain and anxiety states (Schoepp D. D., J. Pharmacol. Exp. Therap. Vol. 299, pages 12-20 (2001)). It appears that group I mGluRs modulate nociceptive transmission or plasticity via modulation of regulated kinases (ERKs) signaling in dorsal horn neurons. Activation of group 1 mGluRs in dorsal horn neurons in response to peripheral inflammation leads to activation of ERK1 and ERK2, resulting in enhanced pain sensitivity (Karim et al., J. Neurosci. Vol. 21, pages 3771-3779, 2001).

There is also evidence that mGluR activation plays a modulatory role in a variety of other normal processes including synaptic transmission, neuronal development, apoptotic neuronal death, synaptic plasticity, spatial learning, olfactory memory, central control of cardiac activity, waking, motor control, and control of the vestibulo-ocular reflex (Nakanishi, Neuron Vol. 13:1031 (1994); Pin et al., Neuropharmacology Vol. 34:1; Knopfel et al., J. Med. Chem. Vol. 38:1417 (1995)).

Because Group I mGluRs activation appears to increase glutamate-mediated neuronal excitation via postsynaptic mechanisms and enhanced presynaptic glutamate release, their activation probably contributes to the pathology of several disorders including degenerative disorders such as senile dementia, Parkinson's disease, Alzheimer's disease, Huntington's Chorea, pain, epilepsy, head trauma, anoxic and ischemic injuries after stroke; psychiatric disorders such as schizophrenia, depression, and anxiety; ophthalmological disorders such as various retinopathies, for example, diabetic retinopathies, glaucoma, and neurological disorders of a auditory nature such as tinnitus, and neuropathic pain disorders, including neuropathic diseases states such as diabetic neuropathies, chemotherapy induced neuropathies, post-herpetic neuralgia, and trigeminal neuralgia; selective mGluR antagonists have been shown to exert anti-dependence activity in vivo (Schoepp et al., Trends Pharmacol. Sci. Vol. 14:13 (1993); Cunningham et al., Life Sci. Vol. 54:135 (1994); Hollman et al., Ann. Rev. Neurosci. Vol. 17:31 (1994); Pin et al., Neuropharmacology Vol. 34:1 (1995); Knopfel et al., J. Med. Chem. Vol. 38:1417 (1995); Tatarczynska et al., Br. Journal of Pharmacology Vol. 132, pages 1423-1430 (2001); Ossowska et al., Neuropharmacology Vol. 41, pages 413-420 (2001); Spooren et al., Trends in Pharmacol. Science. Vol. 22, pages 331-337 (2001); Spooren et al., J. Pharmacol. Exp. Therap. Vol. 295, pages 1267-1275 (2000); Chiamulera et al., Nature Neuroscience Vol. 4, pages 873-874 (2001)).

In view of the above, antagonists to Group I and Group 5 mGlu receptors would be beneficial to treat or ameliorate any of the above-mentioned disorders. Currently available mGluR agonists and antagonists have limited value, due to their lack of potency, limited bioavailability, and poor selectivity. Accordingly, compounds acting as selective antagonists of Group I mGluR receptors may develop as therapeutically beneficial agents, specifically as analgesics, anti-dependence agents, protective agents against degenerative disorders, and anticonvulsants.

### SUMMARY OF THE INVENTION

The present invention discloses compounds, a method for inhibiting the mGlu receptor in mammals using these compounds, a method for controlling pain, neurodegeneration and convulsive states in mammals, and pharmaceutical compositions including those compounds. More particularly, the present invention is directed to compounds of formula (I) 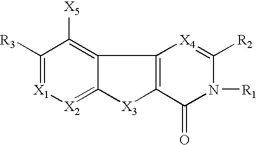
or a pharmaceutically acceptable salt or prodrug thereof, wherein

R₁ is selected from the group consisting of alkyl, aryl, cycloalkyl, heterocycle and heteroaryl;

R₂ is selected from the group consisting of hydrogen and alkyl, R₃ is selected from the group consisting of hydrogen, alkoxyl, aryloxyl, cyano, halogen, heteroalkoxyl, and heteroaryloxyl;

X₁ is selected from the group consisting of —N—, —N⁺(O⁻)— and —C(R₄)—;

X₂ is selected from the group consisting of —N—, —N⁺(O⁻)— and —C(R₅)—;

X₃ is selected form the group consisting of S, O, and NH;

X₄ is selected from the group consisting of N and —C(R₆)—;

X₅ is selected from the group consisting of —NRₐR_{b} and —N⁺(O⁻)RₐR_{b};

R₄ and R₅ are each independently selected from the group consisting of hydrogen, alkyl, haloalkyl, hydroxy, and hydroxyalkyl;

R₆ are each independently selected from the group consisting of hydrogen, alkyl, haloalkyl, hydroxy, and hydroxyalkyl; and

Rₐ and R_{b} are each independently selected from the group consisting of hydrogen, alkyl, alkoxyalkyl, haloalkyl, hydroxyalkyl, arylalkyl, heteroarylalkyl and heterocyclealkyl, R_{c}R_{d}Nalkyl, cyanoalkyl, and cycloalkyl, wherein R_{c} and R_{d} are independently selected from the group consisting of hydrogen and alkyl;

alternatively, Rₐ and R_{b} taken together with the nitrogen to which they are attached form a heterocycle;

with the proviso that

if X₁ is N, then X₂ is —C(R₅)—, and

if X₂ is N, then X₁ is —C(R₄)—; and

the compound is not
9-Dimethylamino-3-(p-tolyl)-3H-5-thia-1,3,6-triazafluoren-4-one;
9-Dimethylamino-3-(p-methoxyphenyl)-3H-5-thia-1,3,6-triazafluoren-4-one;
9-Dimethylamino-3-(p-chlorophenyl)-3H-5-thia-1,3,6-triazafluoren-4-one; and
9-Dimethylamino-3-(p-phenyl)-3H-5-thia-1,3,6-triazafluoren-4-one.

### DETAILED DESCRIPTION OF THE INVENTION

Definition of Terms

As used throughout this specification and the appended claims, the following terms have the following meanings:

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “aryl” as used herein, means a phenyl group, or a bicyclic or a tricyclic fused ring system wherein one or more of the fused rings is a phenyl group. Bicyclic fused ring systems are exemplified by a phenyl group appended to the parent molecular moiety, which is fused to a cycloalkyl group, as defined herein, a phenyl group, a heteroaryl, as defined herein, or a heterocycle as defined herein. Tricyclic fused ring systems are exemplified by an aryl bicyclic fused ring system fused to a cycloalkyl group, as defined herein, a phenyl group, a heteroaryl, as defined herein, or a heterocycle as defined herein. Representative examples of aryl include, but are not limited to, anthracenyl, azulenyl, fluorenyl, indanyl, indenyl, naphthyl, phenyl and tetrahydronaphthyl.

The aryl groups of this invention can be substituted with 0, 1, 2, 3, 4, or 5 substituents independently selected from the group consisting of alkenyl, alkoxy, alkoxyalkyl, alkyl, aryl, carboxy, carboxyalkyl, cyano, cyanoalkyl, formyl, haloalkyl, halogen, heteroaryl, heterocycle, hydroxy, and hydroxyalkyl, wherein the substituent aryl, the heteroaryl and the heterocycle can be substituted with 0, 1, or 2 substitutents independently selected from the group consisting of alkenyl, alkoxy, alkoxyalkyl, alkyl, cyano, cyanoalkyl, formyl, haloalkyl, halogen, hydroxy and hydroxyalkyl. Representative examples include, but are not limited to, 2-bromophenyl, 2-chlorophenyl, 3-chlorophenyl, 4-chlorophenyl, 3-cyanophenyl, 4-cyanophenyl, 2,3-dichlorophenyl, 3,4-dichlorophenyl, 2,5-dichlorophenyl, 2,4-dimethylphenyl, 3,5-dimethylphenyl, 2-fluoro-3-methylphenyl, 2-fluorophenyl, 3-fluorophenyl, 4-fluorophenyl, 2-methoxyphenyl, 3-methoxyphenyl, 4-methoxyphenyl, 2-methylphenyl, 3-methylphenyl, 4-(methylthio)phenyl, 4-nitrophenyl, 4-(trifluoromethoxy)phenyl and 3-(trifluoromethyl)phenyl.

The term “aryloxy,” as used herein, refers to an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “carbonyl,” as used herein, refers to a —C(O)— group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl,” as used herein, refers to a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic fused ring systems are exemplified by a cycloalkyl group appended to the parent molecular moiety, which is fused to an additional cycloalkyl group, as defined herein, a phenyl group, a heteroaryl, as defined herein, or a heterocycle as defined herein. Tricyclic fused ring systems are exemplified by a cycloalkyl bicyclic fused ring system fused to an additional cycloalkyl group, as defined herein, a phenyl group, a heteroaryl, as defined herein, or a heterocycle as defined herein. The additional fused cycloalkyl group may be substituted but may not be fused to another ring. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl ring systems of this invention can be substituted with 0, 1, 2, or 3 substituents independently selected from alkenyl, alkoxy, alkoxyalkyl, alkyl, aryl, carboxy, carboxyalkyl, cyano, cyanoalkyl, formyl, haloalkyl, halogen, heteroaryl, heterocycle, hydroxy, and hydroxyalkyl.

The term “formyl,” as used herein, refers to a —C(O)H group.

The term “halo” or “halogen,” as used herein, refers to —Cl, —Br, —I or —F.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means an aromatic monocyclic ring or an aromatic bicyclic ring. The aromatic monocyclic rings are five or six membered rings wherein 1, 2, 3, or 4 atoms are independently selected from the group consisting of N, O and S. The five membered aromatic monocyclic rings have two double bonds and the six membered aromatic monocyclic rings have three double bonds. The heteroaryl bicyclic rings are exemplified by a heteroaryl monocyclic ring appended to the parent molecular moiety, fused to a phenyl group. The heteroaryl monocyclic rings and the heteroaryl bicyclic rings are connected to the parent molecular moiety through a carbon or nitrogen atom. Representative examples of heteroaryl include, but are not limited to benzothienyl, benzoxadiazolyl, cinnolinyl, dibenzofuranyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, tetrazolyl, thiadiazolyl, thiazolyl, thienopyridinyl, thienyl, triazolyl and triazinyl.

The heteroaryl ring systems of this invention can be substituted with 0, 1, 2, or 3 substituents independently selected from alkenyl, alkoxy, alkoxyalkyl, alkyl, aryl, carboxy, carboxyalkyl, cyano, cyanoalkyl, formyl, haloalkyl, halogen, heteroaryl, heterocycle, hydroxy, and hydroxyalkyl.

The term “heteroarylalkyl” as used herein, refers to a heteroaryl group, as defined herein, appended to the parent molecular moiety through an alkyl group, as defined herein.

The term “heteroaryloxy” as used herein, refers to a heteroaryl group, as defined herein, appended to the parent molecular moiety through an oxygen atom.

The term “heterocycle” or “heterocyclic,” as used herein, refers to a monocyclic, bicyclic, or tricyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. The bicyclic heterocycle rings are composed of a non-aromatic heterocyclic monocyclic ring appended to the parent molecular moiety, which is fused to a cycloalkyl group, as defined herein, or a phenyl group. Alternatively, bicyclic heterocyclic rings are composed of a non-aromatic monocyclic ring fused to another heterocyclic monocyclic ring. Bicyclic ring systems are exemplified by any of the above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, naphthyridinyl, isobenzofuranyl, isobenzothienyl, isoindolyl, isoquinolinyl, phthalazinyl, pyranopyridinyl, quinolinyl, quinolizinyl, quinoxalinyl, quinazolinyl, tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples of tricyclic ring systems include, but are not limited to, acridinyl, carbazolyl, carbolinyl, dibenzo[b,d]furanyl, dibenzo[b,d]thienyl, naphtho[2,3-b]furan, naphtho[2,3-b]thienyl, phenazinyl, phenothiazinyl, phenoxazinyl, thianthrenyl, thioxanthenyl and xanthenyl.

The heterocycle ring systems of this invention can be substituted with 0, 1, 2, or 3 substituents independently selected from alkenyl, alkoxy, alkoxyalkyl, alkyl, aryl, carboxy, carboxyalkyl, cyano, cyanoalkyl, formyl, haloalkyl, halogen, heteroaryl, heterocycle, hydroxy, and hydroxyalkyl.

The term “heterocyclealkyl” as used herein, refers to a heterocycle group, as defined herein, appended to the parent molecular moiety through an alkyl group, as defined herein.

The term “heterocycleoxy” as used herein, refers to a heterocycle group, as defined herein, appended to the parent molecular moiety through an oxygen atom.

The term “hydroxy,” as used herein, refers to an —OH group.

The term “hydroxyalkyl,” as used herein, refers to a hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, and 2-ethyl-4-hydroxyheptyl.

Compounds of the Present Invention

Compounds of the invention can have the formula (I) as described above. More particularly, compounds of formula (I) can include, but are not limited to, compounds wherein X₁ is N—, X₂ is C(R₅), X₃ is O, X₄ is N and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ is N—, X₂ is C(R₅), X₃ is NH, X₄ is N and X₅ is selected from the group consisting of —NRₐR_{b} and —N⁺(O⁻)RₐR_{b}, wherein R₁, R₂ and R₃ are as previously defined for compounds of formula (I).

Preferably, the invention includes those compounds wherein X₁ is N—, X₂ is C(R₅), X₃ is S, X₄ is N and X₅ is selected from the group consisting of —NRₐR_{b} and —N⁺(O⁻)RₐR_{b}, wherein R₁, R₂ and R₃ are as previously defined for compounds of formula (I). More preferably, the invention includes those compounds wherein X₁ is N—, X₂ is C(R₅), X₃ is S, X₄ is N and X₅ is —NRₐR_{b}, R₁ is aryl and R₂ is hydrogen. Most preferably, the invention includes those compounds wherein X₁ is N—, X₂ is C(R₅), X₃ is S, X₄ is N and X₅ is —NRₐR_{b}, Rₐ and R_{b} are selected from the groups alkyl and hydrogen, R₁ is aryl and R₂ is hydrogen. Most preferably, the invention includes those compounds wherein X₁ is N—, X₂ is C(R₅), X₃ is S, X₄ is N and X₅ is —NRₐR_{b}, Rₐ and R_{b} form a heterocycle together with the nitrogen to which they are attached to. Other preferred compounds include those wherein X₁ is N—, X₂ is C(R₅), X₃ is S, X₄ is N, X₅ is —NRₐR_{b}, R₁ is cycloalkyl and, R₂ and R₅ are hydrogen, these include compounds where Rₐ and R_{b} are selected from the group consisting of alkyl and hydrogen. Other preferred compounds include those wherein X₁ is N—, X₂ is C(R₅), X₃ is S, X₄ is N, X₅ is —NRₐR_{b}, Rₐ and R_{b} form a heterocycle together with the nitrogen they arte attached to, R₁ is cycloalkyl and, R₂ and R₅ are hydrogen.

Compounds of the invention can also include those wherein X₁ is N—, X₂ is C(R₅), X₃ is O, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ is N—, X₂ is C(R₅), X₃ is NH, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The invention also includes those compounds wherein X₁ is N—, X₂ is C(R₅), X₃ is S, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ is N⁺(O⁻)—, X₂ is C(R₅), X₃ is O, X₄ is N, and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ is N⁺(O⁻)—, X₂ is C(R₅), X₃ is NH, X₄ is N, and X₅ is selected from the group consisting of —NRₐR_{b} and —N⁺(O⁻)RₐR_{b}, wherein R₁, R₂ and R₃ are as previously defined for compounds of formula (I).

The invention also includes those compounds wherein X₁ is N⁺(O⁻)—, X₂ is C(R₅), X₃ is S, X₄ is N, and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ is N⁺(O⁻)—, X₂ is C(R₅), X₃ is O, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ is N⁺(O⁻)—, X₂ is C(R₅), X₃ is NH, X₄ is C(R₆), and X₅ is selected from the group consisting Of —NRₐR_{b} and —N⁺(O⁻)RₐR_{b}, wherein R₁, R₂ and R₃ are as previously defined for compounds of formula (I).

The invention also includes those compounds wherein X₁ is N⁺(O⁻)—, X₂ is C(R₅), X₃ is S, X₄ is C(R₆), and X₅ is selected from the group consisting Of —NRₐR_{b} and —N⁺(O⁻)RₐR_{b}, wherein R₁, R₂ and R₃ are as previously defined for compounds of formula (I).

Compounds of the invention can also include those wherein X₁ and X₂ are —N⁺(O⁻)—, X₃ is NH, X₄ is N, and X₅ is selected from the group consisting of —NRₐR_{b} and —N⁺(O⁻)RₐR_{b}, wherein R₁, R₂ and R₃ are as previously defined for compounds of formula (I).

The invention also includes those compounds wherein X₁ and X₂ are —N⁺(O⁻)—, X₃ is S, X₄ is N, and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ and X₂ are —N⁺(O⁻)—, X₃ is O, X₄ is N, and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ and X₂ are —N⁺(O⁻)—, X₃ is NH, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The invention also includes those compounds wherein X₁ and X₂ are —N⁺(O⁻)—, X₃ is S, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ and X₂ are —N⁺(O⁻)—, X₃ is O, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The present invention includes compounds wherein X₁ is —C(R₄), X₂ is —N⁺(O⁻)—, X₃ is NH, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The invention also includes those compounds wherein X₁ is —C(R₄), X₂ is —N⁺(O⁻)—, X₃ is S, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

Compounds of the invention can also include those wherein X₁ is —C(R₄), X₂ is —N⁺(O⁻)—, X₃ is O, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

Preferably, the present invention includes compounds wherein X₁ is C(R₄), X₂ is —N⁺(O⁻)—, X₃ is S, X₄ is N, and X₅ is selected from the group consisting of —NRₐR_{b} and —N⁺(O⁻)RₐR_{b}, wherein R₁, R₂ and R₃ are as previously defined for compounds of formula (I). Most preferably, X₅ is NRₐR_{b}, R₁ is aryl and R₂ is hydrogen.

Compounds of the invention also includes those wherein X₁ is —C(R₄), X₂ is —N⁺(O⁻)—, X₃ is O, X₄ is N, and X₅ is selected from the group consisting of —NRₐR_{b} and —N⁺(O⁻)RₐR_{b}, wherein R₁, R₂ and R₃ are as previously defined for compounds of formula (I).

Compounds of the invention also include those wherein X₁ is —C(R₄), X₂ is —N⁺(O⁻)—, X₃ is NH, X₄ is N, and X₅ is selected from the group consisting of —NRₐR_{b} and —N⁺(O⁻)RₐR_{b}, wherein R₁, R₂ and R₃ are as previously defined for compounds of formula (I).

The present invention includes compounds wherein X₁ is —C(R₄), X₂ is N, X₃ is O, X₄ is N, and X₅ is selected from the group consisting of —NRₐR_{b} and —N⁺(O⁻)RₐR_{b}, wherein R₁, R₂ and R₃ are as previously defined for compounds of formula (I).

Compounds also included in the resent invention are those wherein X₁ is —C(R₄), X₂ is N, X₃ is NH, X₄ is N, and X₅ is selected from the group consisting of —NRₐR_{b} and —N⁺(O⁻)RₐR_{b}, wherein R₁, R₂ and R₃ are as previously defined for compounds of formula (I).

The invention preferably includes compounds wherein X₁ is —C(R₄), X₂ is N, X₃ is S, X₄ is N, and X₅ is selected from the group consisting of —NRₐR_{b} and —N⁺(O⁻)RₐR_{b}, wherein R₁, R₂ and R₃ are as previously defined for compounds of formula (I). Most preferably are included compounds wherein X₅ is —NRₐR_{b}, R₁s aryl, and R₂, R₃ and R₄ are hydrogen. Other most preferred compounds included in the present invention are those in which X₁ is —C(R₄), X₂ is N, X₃ is S, X₄ is N, X₅ is —NRₐR_{b}, R₁ is aryl and R₂ is alkyl. Other most preferred compounds included in the present invention are those in which X₁ is —C(R₄), X₂ is N, X₃ is S, X₄ is N, X₅ is —NRₐR_{b}, R₁ is heteroaryll and R₂ is hydrogen. Other most preferred compounds included in the present invention are those in which X₁ is —C(R₄), X₂ is N, X₃ is S, X₄ is N, X₅ is —NRₐR_{b}, R₁ is alkyl and R₂ is hydrogen. Other most preferred compounds included in the present invention are those in which X₁ is —C(R₄), X₂ is N, X₃ is S, X₄ is N, X₅ is —NRₐR_{b}, R₁ is cycloalkyl and R₂ is hydrogen. Other most preferred compounds included in the present invention are those in which X₁ is —C(R₄), X₂ is N, X₃ is S, X₄ is N, X₅ is —NRₐR_{b}, R₁ is heterocycle and R₂ is hydrogen. The present invention also includes compounds in which most preferably X₁ is —C(R₄), X₂ is N, X₃ is S, X₄ is N, X₅ is —NRₐR_{b}, Rₐ and R_{b} are selected from the group consisting of alkyl and hydrogen, R₁ is aryl, R₂, R₃ and R₄ are hydrogen. The present invention also includes compounds in which most preferably X₁ is —C(R₄), X₂ is N, X₃ is S, X₄ is N, X₅ is —NRₐR_{b}, Rₐ and R_{b} form a heterocycle together with the nitrogen to which they are attached, R₁ is aryl, R₂, R₃ and R₄ are hydrogen.

Other preferred compounds include those in which X₁ is —C(R₄), X₂ is N, X₃ is S, X₄ is N, and X₅ is —N⁺(O⁻)RₐR_{b}, wherein Rₐ and R_{b} are selected from the group consisting of alkyl and hydrogen, R₁ is selected from the group consisting of aryl and heterocycle, R₂ and R₃ are as previously defined for compounds of formula (I).

The present invention includes compounds wherein X₁ is C(R₄), X₂ is N, X₃ is O, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The present invention includes compounds wherein X₁ is —C(R₄), X₂ is N, X₃ is S, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The present invention includes compounds wherein X₁ is —C(R₄), X₂ is N, X₃ is NH, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The present invention includes compounds wherein X₁ is C(R₄), X₂ is —C(R₅), X₃ is O, X₄ is N, and X₅ is selected from the group consisting of —NRₐR_{b} and —N⁺(O⁻)RₐR_{b}, wherein R₁, R₂ and R₃ are as previously defined for compounds of formula (I).

Compounds also included in the resent invention are those wherein X₁ is —C(R₄), X₂ is —C(R₅), X₃ is NH, X₄ is N, and X₅ is selected from the group consisting of —NRₐR_{b} and —N⁺(O⁻)RₐR_{b}, wherein R₁, R₂ and R₃ are as previously defined for compounds of formula (I).

The invention preferably includes compounds wherein X₁ is —C(R₄), X₂ is —C(R₅), X₃ is S, X₄ is N, and X₅ is selected from the group consisting of —NRₐR_{b} and —N⁺(O⁻)RₐR_{b}, wherein R₁, R₂ and R₃ are as previously defined for compounds of formula (I). Most preferably are included compounds wherein X₅ is —NRₐR_{b}, R₁ is aryl, and R₂ is hydrogen.

The present invention includes compounds wherein X₁ is —C(R₄), X₂ is −5), X₃ is O, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The present invention includes compounds wherein X₁ is —C(R₄), X₂ is —C(R₅), X₃ is S, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

The present invention includes compounds wherein X₁ is —C(R₄), X₂ is —C(R₄), X₃ is NH, X₄ is C(R₆), and X₅ is selected from the group consisting of —NRₐR_{b} and —N⁺(O⁻)RₐR_{b}, wherein R₁, R₂ and R₃ are as previously defined for compounds of formula (I).

Preparation of Compounds of the Present Invention

The compounds and processes of the present invention will be better understood in connection with the following synthetic Schemes and Examples that illustrate a means by which the compounds of the present invention can be prepared.

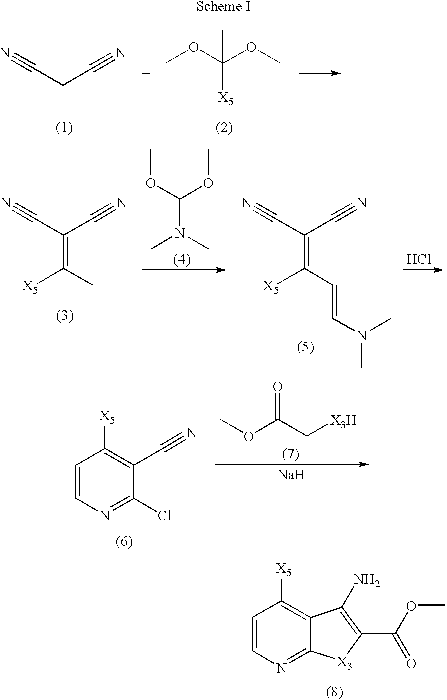

As demonstrated in Scheme 1, compounds of formula (1) when treated with compounds of formula (2) will provide compounds of formula (3). Compounds of formula (3) when treated with compounds of formula (4) under heated conditions will provide compounds of formula (5). Compounds of formula (5) when treated to hydrochloric acid will provide compounds of formula (6). A solution of compound of formula (7) is treated with sodium hydride followed by treatment with the compounds of formula (6) will provide a compound of formula (8).

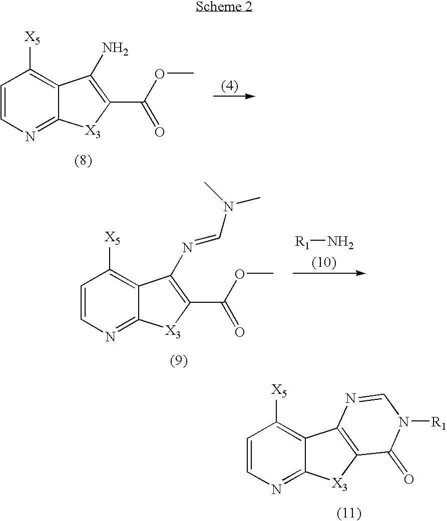

As outlined in Scheme 2, compounds of formula (8) when treated with compounds of formula (4) under heated conditions will provide compounds of formula (9). Compounds of formula (9) when treated with amines of formula (10) under heated conditions will provide compounds of formula (11), which are representative of compounds of the present invention.

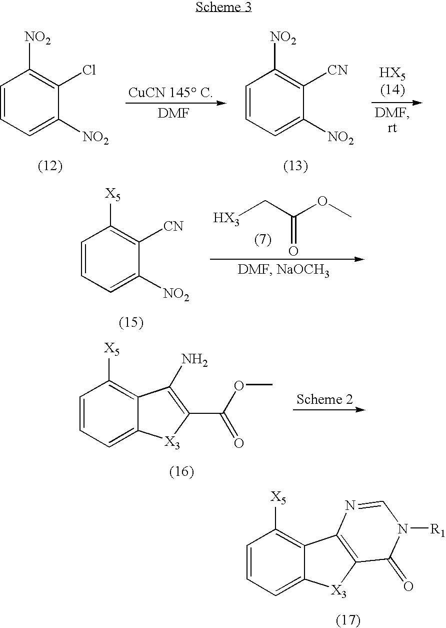

As outlined in Scheme 3, compounds of formula (12) when treated with copper cyanide under heated conditions in DMF will provide compounds of formula (13). Compounds of formula (13) when treated with compounds of formula (14) wherein X₅ is NRₐR_{b} in DMF will provide compounds of formula (15). Compounds of formula (15) when treated with compounds of formula (7) and a base such as but limited to sodium methoxide in DMF will provide compounds of formula (16). Compounds of formula (16) when subjected to the conditions outlined in Scheme 2 will provide compounds of formula (17) which are representative compounds of the present invention.

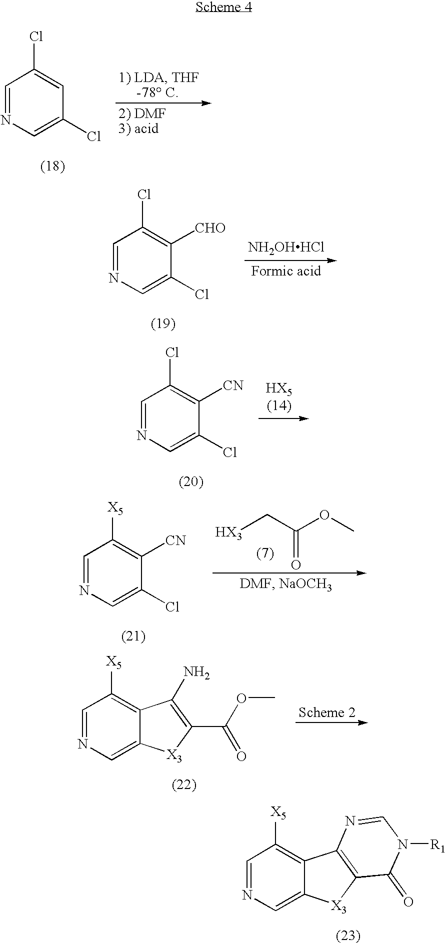

As outlined in Scheme 4, dichloride compounds of formula (18) when treated with lithium diisopropylamide at −78 C. in THF, followed by addition of DMF followed by an acidic workup will provide aldehydes of formula (19). Compounds of formula (19) when treated with hydroxylamine hydrochloride and formic acid in concentrated sulfuric acid under heated conditions will provide nitrites of formula (20). Compounds of formula (20) when treated with compounds of formula (14) wherein X₅ is NRₐR_{b} in DMF will provide compounds of formula (21). Compounds of formula (21) when treated with compounds of formula (7) which was pretreated with a base such as but not limited to sodium methoxide in solvents such as but not limited to DMF will provide compounds of formula (22). Compounds of formula (22) when treated according to the procedure outlined in Scheme 2 will provide compounds of formula (23) which are representative of the present invention.

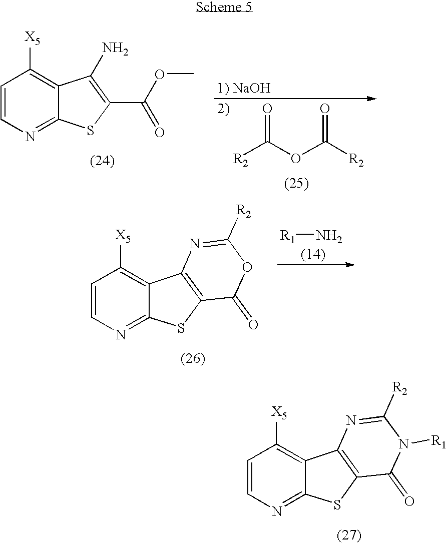

As outlined in Scheme 5, compounds of formula (24) is treated with sodium hydroxide to form the sodium salt followed by treatment with an anhydride of formula (25) will provide compounds of formula (26). Similarly, the corresponding carboxylic acid form of compounds of formula (24) may also be treated to the same conditions without necessitating the hydrolysis step. Compounds of formula (26) when treated with compounds of formula (14) will provide compounds of formula (27) which are representative compounds of the present invention.

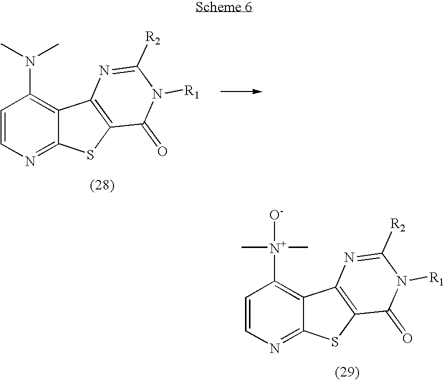

As outlined in Scheme 6, compounds of formula (28) when treated with a mixture of phthalic anhydride and hydrogen peroxide-urea complex will provide compounds of formula (29), which are representative of the compounds of the present invention.

Compositions of the Invention

The invention also provides pharmaceutical compositions comprising a therapeutically effective amount of a compound of formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention. The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The compounds of the invention can be used in the form of pharmaceutically acceptable salts, esters, or amides derived from inorganic or organic acids. The term “pharmaceutically acceptable salts, esters and amides,” as used herein, include salts, zwitterions, esters and amides of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term “pharmaceutically acceptable ester,” as used herein, refers to esters of compounds of the invention which hydrolyze in vivo and include those that break down readily in the human body to leave the parent compound or a salt thereof. Examples of pharmaceutically acceptable, non-toxic esters of the invention include C₁-to-C₆ alkyl esters and C₅-to-C₇ cycloalkyl esters, although C₁-to-C₄ alkyl esters are preferred. Esters of the compounds of formula (I) can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl trifilate, for example with methyl iodide, benzyl iodide, cyclopentyl iodide. They also can be prepared by reaction of the compound with an acid such as hydrochloric acid and an alkylcarboxylic acid such as acetic acid, or with acid and an arylcarboxylic acid such as benzoic acid.

The term “pharmaceutically acceptable amide,” as used herein, refers to non-toxic amides of the invention derived from ammonia, primary C₁-to-C₆ alkyl amines and secondary C₁-to-C₆ dialkyl amines. In the case of secondary amines, the amine can also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I) can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also can be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds of formula (I).

Methods of the Invention

Compounds and compositions of the invention are useful for modulating the effects resulting from stimulation of mGluRs, and more particularly Group I mGluRs. Specifically, the compounds and compositions of the invention can be used for treating and preventing disorders modulated by mGluRs. Typically, such disorders can be ameliorated by selectively modulating the mGluRs in a mammal, preferably by administering a compound or composition of the invention, either alone or in combination with another active agent, for example, as part of a therapeutic regimen.

The compounds of the invention, including but not limited to those specified in the examples, possess an affinity for, and are able to block, mGluRs and more particularly Group I mGluRs. As mGluRs antagonists, the compounds of the invention can be useful for the treatment and prevention of a number of mGluRs-mediated diseases or conditions.

For example, mGluRs have been shown to play a significant role in the etiology of disorders such as epilepsy, focal and global ischemia, pain and neurodegeneration (Knopfel et al., J. Med. Chem. Vol. 38, pages 1417-1426, 1995). Epilepsy can result form excessive glutamatergic activation. Several lines of evidence suggest the possible therapeutic value of antagonists of mGlu receptors in inhibiting said excessive glutamatergic transmission. More specifically, mGluRs antagonists have been shown to protect mice against audiogenic tonic and clonic convulsions resulting from excessive excitatory amino acid release (Thomsen et al., J. Neurochem. Vol. 62, pages 2492-2495, 1994).

Glutamate is one of the amino acids present in the brain that mediates excitotoxicity. Pathological changes seen in animal models subjected to glutamatergic stimulation are similar to pathological changes seen in brain after ischemic attacks (Choi D W, Trends in Neurosciences Vol. 11, pages 465-469, 1988). Studies like the foregoing indicate the potential therapeutic utility of Group I mGluRs antagonists in protecting brain tissue against the damages resulting from abnormal physiological glutamate receptor activation. mGLuR antagonists were able to reduce akinesia and muscle rigidity in animal models with induced Parkinsonian symptoms (Ossowska et al., Neuropharmacology Vol. 41, pages 413-420 (2001); Spooren et al., Trends in Pharmacol. Science. Vol. 22, pages 331-337 (2001). Therefore, antagonists of mGlu receptors may become very important tool in the treatment of parkinsonian symptoms.

Antagonists of the mGluRs have demonstrated a very broad and potent anxyolitic activity in male rodent models of anxiety, in the so-called conditioned response tests. Antidepressant-like effects of mGluRs antagonists were also observed in male rats in several tests (Tatarczynska et al., Br. Journal of Pharmacology Vol. 132, pages 1423-1430 (2001); Spooren et al., J. Pharmacol. Exp. Therap. Vol. 295, pages 1267-1275 (2000)).

MGluRs are involved in the behavioral effects of psychostimulants such as cocaine. Studies in wild type and mutant mice, which lack mGluR5 expression, have shown that reinforcing locomotor stimulant effects of cocaine are absent in mutant mice. These have suggested an essential role of mGluR5 in cocaine self-administration and locomotor effects, therefore the importance of mGluR antagonists in the treatment of drug-dependence (Chiamulera et al., Nature Neuroscience Vol. 4, pages 873-874 (2001).

Noxious stimuli appear to be modulated through group I mGluRs via modulation of regulated kinases signaling in dorsal horn neurons. Group I mGluRs activation in dorsal horn neurons in response to peripheral inflammation results in enhanced pain sensitivity in mice (Karim et al., J. Neuroscience Vol. 21, pages 3771-3779, 2001). Therefore, antagonists of group I mGluRs are potential therapeutic agents useful for the treatment of pain states, including acute pain, post-surgical pain, as well as chronic pain states including inflammatory pain and neuropathic pain.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal range from about 0.10 mg/kg body weight to about 1 g/kg body weight. More preferable doses can be in the range of from about 0.10 mg/kg body weight to about 100 mg/kg body weight. If desired, the effective daily dose can be divided into multiple doses for purposes of administration. Consequently, single dose compositions may contain such amounts or submultiples thereof to make up the daily dose.

The compounds and processes of the invention will be better understood by reference to the following examples and reference examples, which are intended as an illustration of and not a limitation upon the scope of the invention.

### EXAMPLES

The following Examples are intended as an illustration of and not a limitation upon the scope of the invention as defined in the appended claims.

### Example 1

### 9-Dimethylamino-3-(o-tolyl)-3H-5-thia-1,3,6-triazafluoren-4-one

### Example 1A

### 2-(1-Dimethylaminoethylidene)-malononitrile

To a solution of N,N-Dimethylacet-amide dimethyl acetal (90%, 125.0 g, 845 mmol) in ethanol (75 mL), a solution of malononitrile (56.0 g, 847.2 mmol) in diethyl ether (500 mL) was added slowly at 0° C. The reaction mixture was then gradually warmed up to room temperature and stirred for 2 days. Solvent was removed, and the solid was re-dissolved in ethyl acetate, and was purified via a short column chromatography (SiO₂, ethyl acetate) to give a pale yellow solid product (109.2 g, 96%). 1H NMR (300 MHz, CDCl3) δ 3.32 ppm (s, br, 6 H); 2.28 (s, 3H). M/Z (ESI, M+1): 135.9.

### Example 1B

### 2-[1,3-Bis(dimethylaminoallylidene)]malononitrile

Example 1A (100.0 g, 740 mmol) was dissolved in N,N-dimethylformamide dimethyl acetal (94%, 210 mL, 1.48 mol), and the reaction mixture was heated to reflux at 100° C. for 1 hours 20 min. Cooled down to room temperature. Solid was collected, and washed with cold methanol (10×3 mL) to give a yellow solid product. The mother liquor was concentrated, and the solid was collected again, and washed with cold methanol. This procedure was repeated couple of times, and all the solid products were combined (113.7 g, 81%). The final residue was purified by a short column chromatography (SiO₂, ethyl acetate) to give additional 17.2 g product (12%). 1H NMR (300 MHz, CDCl3) δ 7.42 ppm (d, J=12.2 Hz, 1H); 4.36 (d, J=12.2 Hz, 1H); 3.15 (s, 6H), 3.05 (s, br, 6H). M/Z (ESI, M+1): 191.1.

### Example 1C

### 4-Dimethylamino-2-chloro-3-cyanopyridine

To a slurry of 2-(1,3-bisdimethylamino-allylidene)malononitrile (120.0 g, 632 mmol) (Example 1B) in methanol (1500 mL), HCl gas was introduced gently at 0° C. The reaction mixture became homogeneous in about 1 hr, and was allowed to stir under constant HCl flow for additional 9 hours at 0° C. N2 was bubbled though the reaction mixture for 2 hr., and all the solvent was removed. The residue solid was re-dissolved in CH₂Cl₂, and washed with water/K₂CO₃/water. Organic layer was separated and dried over Na₂SO₄. Removal of salt and solvent gave a pure product (113.0 g, 98%). ¹H NMR (300 MHz, CDCl₃) δ ¹H NMR (300 MHz, CDCl₃) δ 7.96 ppm (d, J=6.4 Hz, 1H); 6.59 (d, J=6.4 Hz, 1H); 3.30 (s, 6H). M/Z (ESI, M+1): 181.9.

### Example 1D

### 5-Amino-4-dimethylaminothieno[8,9-b]pyridine-6-carboxylic acid methyl ester

To a solution of methyl thioglycolate (40 mL, 442 mmol) in anhydrous THF (500 mL), sodium hydride (60%, 20.0 g, 500 mmol) was added in small portions at 0° C. A solution of 4-dimethylamino-2-chloro-3-cyanopyridine (80.0 g, 442 mmol) (from step 3) in THF (1000 mL) was added. The reaction mixture was allowed to stir for 1 day at room temperature. Additional NaH (60%, 11.0 g, 276 mmol) was added, and the reaction mixture was then heated to reflux for 1 hour for 50 minutes. After cooled down to room temperature, the reaction mixture was quenched with saturated NH₄Cl. Organic solvent was removed under vacuum, and the aqueous layer was extracted with dichloromethane (500 mL). Organic layer was separated and washed with water and brine, then dried over Na₂SO₄. The solvent was concentrated, and a pure product was crystallized. Solid was collected and washed with cold methanol several times, then ether, and dried over the air (82.3 g, 74%). Additional solid was collected via the same, repeated procedures (12.1 g, 11%). ¹H NMR (300 MHz, CDCl₃) δ 8.43 ppm (d, J=5.2 Hz, 1H), 6.83 (d, J=5.2 Hz, 1H), 6.74, s, br, 2H), 3.86 (s, 3H), 2.84 (s, 6H). M/Z (ESI, M+1): 251.9.

### Example 1E

### 4-Dimethylamino-5-(dimethylaminomethyleneamcinothieno)[8,9-b]pyridine-6-carboxylic acid methyl ester

5-Amino-4-dimethylaminothieno[8,9-b]pyridine-6-carboxylic acid methyl ester (32.0 g, 127 mmol) (from step 4) was dissolved in ethanol (150 mL) and N,N-dimethylformamide dimethyl acetal (100 mL), and heated to reflux for 4.5 h. Excess of solvent and reagent were removed to give a yellow solid product (38.5 g, 99%). ¹H NMR (300 MHz, CDCl₃) δ 8.30 ppm (d, J=5.4 Hz, 1H), 7.41 (s, 1H), 6.60 (d, J=5.4 Hz, 1H), 3.82 (s, 3H), 3.17 (s, br, 3H), 3.07 (s, br, 3H), 2.99 (s, 6H). M/Z (ESI, M+1): 307.0.

### Example 1F

### 9-Dimethylamino-3-(o-tolyl)-3H-5-thia-1,3,6-triazafluoren-4-one

4-Dimethylamino-5-(dimethylaminomethyleneamino)thieno[8,9-b]pyridine-6-carboxylic acid methyl ester (1.0 g, 3.2 mmol) (Example 1E), para-toluenesulfonic acid (25 mg, 0.13 mmol) and o-toluidine (512 μL, 4.8 mmol) were placed in flask with toluene (25 mL) and then heated to 130 C. for over night. Cooled down to room temperature. Solvent was removed under vacuum, and the residue was treated with cold methanol following sonication. White precipitate was formed. The product was then collected, and washed with cold methanol, and dried under vacuum to give a pure product (451 mg, 42%). 1H NMR (300 MHz, CDCl3/MeOD) δ 8.43 ppm (d, J=6.6 Hz, 1H), 8.19 (s, 1H), 7.43 (m, 3H), 7.28 (d, J=7.8 Hz, 1H), 6.89 (d, J=6.6 Hz, 1H), 3.37 (s, 6H), 2.21 (s, 3H). M/Z (ESI, M+I): 337.0.

### Example 2

### 9-Dimethylamino-3-(m-tolyl)-3H-5-thia-1,3,6-triazafluoren-4-one

Compound was prepared by procedure described for Example 1 substituting o-toluidine with m-toluidine. ¹H NMR (300 MHz, CDCl₃/MeOD) δ 8.43 ppm (d, J=5.6 Hz, 1H), 8.29 (s, 1H), 7.46 (t, J=7.8 Hz, 1H), 7.34 (d, J=7.8 Hz, 1H), 7.28 (s, 1H), 7.25 (d, J=7.8 Hz, 1H), 6.84 (d, J=5.6 Hz, 1H), 3.25 (s, 6H), 2.46 (s, 3H). M/Z (ESI, M+1): 336.9.

### Example 4

### 9-Dimethylamino-3-(o-hydroxyphenyl)-3H-5-thia-1,3,6-triazafluoren-4-one

Compound was prepared by procedure described for Example 1 substituting o-toluidine with o-hydroxyaniline. ¹H NMR (300 MHz, CDCl₃/MeOD) δ 8.41 ppm (d, J=5.9 Hz, 1H), 8.27 (s, 1H), 7.39 (m, 1H), 7.28 (m, 1H), 7.11 (m, 1H), 7.05 (m, 1H), 6.83 (d, J=5.9 Hz, 1H), 3.26 (s, 6H). M/Z (ESI, M+1): 338.9.

### Example 5

### 9-Dimethylamino-3-(m-fluorophenyl)-3H-5-thia-1,3,6-triazafluoren-4-one

Compound was prepared by procedure described for Example 1 substituting o-toluidine with m-fluoroaniline¹H NMR (300 MHz, CDCl₃/MeOD) δ 8.44 ppm (d, J=6.2 Hz, 1H), 8.29 (s, 1H), 7.56 (m, 1H), 7.26 (m, 3H), 6.86 (d, J=6.2 Hz, 1H), 3.29 (s, 6H). M/Z (ESI, M+1): 340.9.

### Example 6

### 9-Dimethylamino-3-(p-fluorophenyl)-3H-5-thia-1,3,6-triazafluoren-4-one

Compound was prepared by procedure described for Example 1 substituting o-toluidine with p-fluoroaniline. ¹H NMR (300 MHz, CDCl₃/MeOD) δ 8.43 ppm (d, J=5.9 Hz, 1H), 8.28 (s, 1H), 7.46 (m, 2H), 7.27 (m, 2H), 7.36 (m, 2H), 6.84 (d, J=5.9 Hz, 1H), 3.25 (s, 6H). M/Z (ESI, M+1): 340.9.

### Example 7

### 9-Dimethylamino-3-(m-chlorophenyl)-3H-5-thia-1,3,6-triazafluoren-4-one

Compound was prepared by procedure described for Example 1 substituting o-toluidine with m-chloroaniline. ¹H NMR (300 MHz, CDCl₃/MeOD) δ 8.43 ppm (d, J=6.2 Hz, 1H), 8.27 (s, 1H), 7.52 (m, 2H), 7.47 (m, 1H), 7.38 (m, 1H), 6.84 (d, J=6.2 Hz, 1H), 3.23 (s, 6H). M/Z (ESI, M+1): 356.9.

### Example 8

### 9-Dimethylamino-3-(p-bromophenyl)-3H-5-thia-1,3,6-triazafluoren-4-one

Compound was prepared by procedure described for Example 1 substituting o-toluidine with p-bromoaniline. ¹H NMR (300 MHz, CDCl₃/MeOD) δ 8.43 ppm (d, J=5.9 Hz, 1H), 8.26 (s, 1H), 7.72 (d, J=8.7 Hz, 2H), 7.36 (d, J=8.7 Hz, 2H), 6.84 (d, J=5.9 Hz, 1H), 3.26 (s, 6H). M/Z (ESI, M+1): 400.0, 402.8.

### Example 9

### 9-Dimethylamino-3-(p-trifluoromethylphenyl)-3H-5-thia-1,3,6-triazafluoren-4-one

Compound was prepared by procedure described for Example 1 substituting o-toluidine with p-trifluoromethylaniline¹H NMR (300 MHz, CDCl₃/MeOD) δ 8.45 ppm (s, br, 1H), 8.29 (s, 1H), 7.87 (d, J=8.4 Hz, 2H), 7.64 (d, J=8.4 Hz, 2H), 6.85 (d, J=5.3 Hz, 1H), 3.23 (s, 6H). M/Z (ESI, M+1): 390.9.

### Example 10

### 9-Dimethylamino-3-(2,4-dimethylphenyl)-3H-5-thia-1,3,6-triazafluoren-4-one

Compound was prepared by procedure described for Example 1 substituting o-toluidine with 2,4-dimethylaniline. ¹H NMR (300 MHz, CDCl₃/MeOD) δ 8.44 ppm (d, J=5.9 Hz, 1H), 8.17 (s, 1H), 7.23 (s, 1H), 7.18 (d, J=8.1 Hz, 1H), 7.15 (d, J=8.1 Hz, 1H), 6.85 (d, J=5.9 Hz, 1H), 3.28 (s, 6H), 2.42 (s, 3H), 2.17 (s, 3H). M/Z (ESI, M+1): 351.0.

### Example 11

### 9-Dimethylamino-3-(3,4-methylenedioxyphenyl)-3H-5-thia-1,3,6-triazafluoren-4-one

Compound was prepared by procedure described for Example 1 substituting o-toluidine with 3,4-methylenedioxyaniline. ¹H NMR (300 MHz, CDCl₃/MeOD) δ 8.43 ppm (d, J=6.2 Hz, 1H), 8.27 (s, 1H), 6.96 (d, J=8.1 Hz, 1H), 6.95 (d, J=2.5 Hz, 1H), 6.87 (dd, J=8.1, 2.5 Hz, 1H), 6.85 (d, J=6.2 Hz, 1H), 6.09 (s, 2H), 3.28 (s, 6H). M/Z (ESI, M+1): 366.9.

### Example 12

### 9-Dimethylamino-3-(2,4-dichlorophenyl)-3H-5-thia-1,3,6-triazafluoren-4-one

Compound was prepared by procedure described for Example 1 substituting o-toluidine with 2,4-dichloroaniline. ¹H NMR (300 MHz, CDCl₃/MeOD) δ 8.42 ppm (d, J=6.2 Hz, 1H), 8.12 (s, 1H), 7.67 (d, J=2.2 Hz, 1H), 7.48 (dd, J=8.7, 2.2 Hz, 1H), 7.44 (d, J=8.7 Hz, 1H), 6.87 (d, J=6.2 Hz, 1H), 3.33 (s, 6H). M/Z (ESI, M+1): 390.9.

### Example 13

### 9-Dimethylamino-3-(2,5-dichlorophenyl)-3H-5-thia-1,3,6-triazafluoren-4-one

Compound was prepared by procedure described for Example 1 substituting o-toluidine with 2,5-dichloroaniline. ¹H NMR (300 MHz, CDCl₃/MeOD) δ 8.44 ppm (d, J=6.2 Hz, 1H), 8.12 (s, 1H), 7.59 (m, 1H), 7.52 (m, 2H), 6.86 (d, J=6.2 Hz, 1H), 3.29 (s, 6H). M/Z (ESI, M+1): 390.9.

### Example 14

### 9-Dimethylamino-3-(thiozol-2-yl)-3H-5-thia-1,3,6-triazafluoren-4-one

Compound was prepared by procedure described for Example 1 substituting o-toluidine with 2-aminothiozole. ¹H NMR (300 MHz, CDCl₃/MeOD) δ 9.66 ppm (s, 1H), 8.39 (d, J=5.9 Hz, 1H), 7.78 (d, J=3.4 Hz, 1H), 7.46 (d, J=3.4 Hz, 1H), 6.85 (d, J=5.9 Hz, 1H), 3.25 (s, 6H). M/Z (ESI, M+1): 329.9.

### Example 15

### 9-Dimethylamino-3-(2′,2′-dimethyl-1′-propyl)-3H-5-thia-1,3,6-triazafluoren-4-one

Compound was prepared by procedure described for Example 1 substituting o-toluidine with 2,2-dimethylpropylamine. ¹H NMR (CDCl₃/MeOD, 300 MHz): δ 8.42 ppm (d, J=6.2 Hz, 1H), 8.18 (s, 1H), 6.84 (d, J=6.2 Hz, 1H), 3.97 (s, 2H), 3.29 (s, 6H), 1.05 (s, 9H). M/Z (ESI, M+1): 317.0.

### Example 16

### 9-Dimethylamino-3-(4-ethylphenyl)-3H-5-thia-1,3,6-triazafluoren-4-one

Compound was prepared by procedure described for Example 1 substituting o-toluidine with p-ethylaniline. ¹H NMR (300 MHz, CDCl₃/MeOD) δ 8.43 ppm (d, J=5.9 Hz, 1H), 8.29 (s, 1H), 7.40 (d, J=8.4 Hz, 2H), 7.36 (d, J=8.4 Hz, 2H), 6.84 (d, J=5.9 Hz, 1H), 3.27 (s, 6H), 2.76 (q, J=7.8 Hz, 2H), 1.30 (t, 7.8 Hz, 3H). M/Z (ESI, M+1): 351.0.

### Example 17

### 9-Dimethylamino-3-(3-fluoro-4-methylphenyl)-3H-5-thia-1,3,6-triazafluoren-4-one

Compound was prepared by procedure described for Example 1 substituting o-toluidine with 3-fluoro-4-methylaniline. ¹H NMR (300 MHz, CDCl₃/MeOD) δ 8.43 ppm (d, J=5.3 Hz, 1H), 8.27 (s, 1H), 7.39 (t, J=8.1 Hz, 1H), 7.19 (m, 1H), 7.17 (m, 1H), 6.83 (d, J=5.3 Hz, 1H), 3.27 (s, 6H), 2.38 (s, 3H). M/Z (ESI, M+1): 354.9.

### Example 18

### 9-Dimethylamino-3-(4-fluoro-2-methylphenyl)-3H-5-thia-1,3,6-triazafluoren-4-one

Compound was prepared by procedure described for Example 1 substituting o-toluidine with 4-fluoro-2-methylaniline. ¹H NMR (300 MHz, CDCl₃/MeOD) δ 8.44 ppm (d, J=6.2 Hz, 1H), 8.16 (s, 1H), 7.27 (m, 1H), 7.14 (m, 1H), 7.09 (m, 1H), 6.87 (d, J=6.2 Hz, 1H), 3.34 (s, 6H), 2.20 (s, 3H). M/Z (ESI, M+1): 354.9.

### Example 19

### 9-Dimethylamino-3-(N-hexamethyleneiminyl)-3H-5-thia-1,3,6-triazafluoren-4-one

Compound was prepared by procedure described for Example 1 substituting o-toluidine with 1-aminohomopiperidine. ¹H NMR (300 MHz, CDCl₃/MeOD) δ 8.40 ppm (d, J=5.8 Hz, 1H), 8.37 (s, 1H), 6.78 (d, J=5.8 Hz, 1H), 3.88 (m, 4H), 3.15 (s, 6H), 1.78 (m, 8H). M/Z (ESI, M+1): 344.1.

### Example 20

### 9-Dimethylamino-3-(2′-methoxy-5′-pyridinyl)-3H-5-thia-1,3,6-triazafluoren-4-one

Compound was prepared by procedure described for Example 1 substituting o-toluidine with 5-amino-2-methoxypyridine. ¹H NMR (300 MHz, CDCl₃/MeOD) δ 8.44 ppm (d, J=5.8 Hz, 1H), 8.25 (s, 1H), 8.23 (d, J=2.7 Hz, 1H), 7.75 (dd, J=8.8, 2.7 Hz, 1H), 6.93 (d, J=8.8 Hz, 1H), 6.81 (d, J=5.8 Hz, 1H), 4.01 (s, 3H), 3.18 (s, 6H), 1.78 (m, 8H). M/Z (ESI, M+1): 354.0.

### Example 21

### 9-(dimethylamino)-3-(4-ethylphenyl)[1]benzothieno[3,2-d]pyrimidin-4(3H)-one

Compound was prepared using the procedure described in Example 22 substituting 2-nitro-6-dimethylamino-benzonitrile for 3-dimethylamino-4-cyano-5-chloropyridine in step 3 to provide the title compound (13%) as beige color solid. ¹H NMR (300 MHz, DMSO-d₆) δ 1.25 (t, J=9 Hz, 3H), 2.72 (q, J=9 Hz, 2H), 2.95 (s, 6H), 7.07 (d, J=9 Hz, 1H), 7.51 (m, 5H), 7.65 (d, J=9 Hz, 1H), 8.58 (s, 1H); M/Z (DCI/NH₃) 350 (M+H)⁺. Anal. calcd for C₂₀H₁₉N₃OS: C, 68.74; H, 5.48; N, 12.02. Found: C, 68.19; H, 5.74; N, 12.34.

### Example 22

### 9-(dimethylamino)-3-(4-ethylphenyl)pyrido[4′,3′:4,5]thieno[3,2-d]pyrimidin-4(3H)-one

### Example 22A

### 3,5-dichloro-4-pyridinecarboxaldehyde

A mixture of 3,5-dichloro-4-pyridinecarboxaldehyde (10.0 g, 57.1 mmol), hydroxylamine hydrochloride (5.25 g 76 mmol), formic acid (50 ml) and H₂SO₄ conc. (5 drops) was refluxed under N₂ for 6 hours The mixture was concentrated under vacuum. The solids were taken in Et₂O (250 ml) washed with NaHCO₃, brine, organics dried with MgSO₄, filtered, concentrated, recrystallized from hexane to give 8.2 g (83%) of desired nitrile.m.p.114° C.

### Example 22B 3-dimethylamino-4-cyano-5-chloropyridine

The 3,5 dichloro-4-cyanopyridine from above (3.0 g, 17.2 mmol) was dissolved in DMF (25 ml). The mixture was cooled to 0° C. To this mixture was added 40% aqueous dimethylamine (7.0 ml, 35 mmol). The mixture was allowed to come to room temperature and stirred at that temperature for 4 hours and then poured into ice water. The precipitate formed was filtered, vacuum dried to obtain 2.3 g (75%) of desired nitrile as beige color solid. m.p.114° C.

### Example 22C

### 3-amino-4-dimethylamino-thieno[2,3]pyridine-2-carboxylic acid methyl ester

A mixture of 3-dimethylamino-4-cyano-5-chloropyridine from step 2 (1.9 g, 10.4 mmol), methyl thioglycolate (1.2 g 10.4 mmol) was dissolved in dry DMF (20 ml). The mixture was cooled to 0° C. Sodium methoxide (1.2 g, 22 mmol) was then added under N₂. The reaction mixture was allowed to come to room temperature and allowed to stir for 16 hours, poured into ice water (250 ml), yellow precipitate formed was filtered, vacuum dried to obtain 2.3 g (88%) of desired amino carboxylate as yellow amorphous solid.

### Example 22D

### 4-dimethylamino-3-(dimethylamino-methyleneamino)-thieno[2,3]pyridine-2-carboxylic acid methyl ester

A mixture of amino carboxylate (1.9 g, 7.6 mmol) from step 3, dimethylformamide dimethylacetal (6.0 ml), EtOH (6 ml) was refluxed under N₂ for 16 hours, cooled to room temperature, concentrated under vacuum. The solid obtained was recrystallized from EtOH-water to obtain 2.1 g (90%) of desired carboxylate.

### Example 22E

### 9-(dimethylamino)-3-(4-ethylphenyl)pyrido[4′,3′:4,5]thieno[3,2-d]pyrimidin-4(3H)-one

A mixture of carboxylate (0.306 g, 1.0 mmol) from step-4 above, 4-ethyl aniline (0.181 g, 1.5 mmol), p-toluene sulfonic acid (0.02 g, 0.1 mmol) and toluene (7 ml) was refluxed under N₂ for 16 hours. Reaction mixture was cooled to room temperature, concentrated under vacuum, residue purified by flash column chromatography (silica gel, 1:1. Hexane:EtOAc) to give 0.11 g (38%) of desired pyrimidone as yellow color solid. 1H NMR (300 MHz, DMSO-d6) δ 1.24 (t, J=9 Hz, 3H), 2.71 (q, J=9 Hz, 2H), 3.01 (s, 6H), 7.42 (d, J=9 Hz, 2H), 7.50 (d, J=9 Hz, 2H), 8.27 (s, 1H), 8.63 (s, 1H), 8.96 (s, 1H); M/Z (DCI/NH3) 351 (M+H)+. Anal. calcd for C19H18N4OS: C, 65.12; H, 5.18; N, 15.99. Found: C, 64.94; H, 5.03; N, 15.88.

### Example 23

### 9-(dimethylamino)-3-(3-fluoro-4-methylphenyl)pyrido[4′,3′:4,5]thieno[3,2-d]pyrimidin-4(3H)-one

Compound was prepared using the procedure described in Example 22 substituting 3-fluoro-4-methyl aniline for 4-ethyl aniline to provide the title compound (51%) as yellow color solid. ¹H NMR (300 MHz, DMSO-d₆) δ 2.38 (d, J=1.5 Hz, 3H), 3.02 (s, 6H), 7.38 (m, 1H), 7.52 (m, 2H), 8.27 (s, 1H), 8.63 (s, 1H), 8.97 (s, 1H); M/Z (DCI/NH₃) 355 (M+H)⁺. Anal. calcd for C₁₈H₁₅FN₄OS: C, 61.00; H, 4.27; N, 15.81. Found: C, 61.29; H, 4.64; N, 16.09.

### Example 24

### 9-(dimethylamino)-3-(4-methylphenyl)pyrido[4′,3′:4,5]thieno[3,2-d]pyrimidin-4(3H)-one

Compound was prepared using the procedure described in Example 22 substituting p-toluidine for 4-ethyl aniline to provide the title compound (58%) as yellow color solid. ¹H NMR (300 MHz, DMSO-d₆) δ 2.42 (s, 3H), 3.02 (s, 6H), 7.40 (d, J=9 Hz, 2H), 7.46 (d, J=9 Hz, 2H), 8.27 (s, 1H), 8.63 (s, 1H), 8.97 (s, 1H); M/Z (DCI/NH₃) 337 (M+H)⁺. Anal. calcd for C₁₈H₁₆N₄OS: C, 61.02; H, 5.08; N, 15.82. Found: C, 60.95; H, 4.75; N, 15.70.

### Example 25

### 3-cycloheptyl-9-(dimethylamino)pyrido[4′,3′:4,5]thieno[3,2-d]pyrimidin-4(3H)-one

Compound was prepared using the procedure described in Example 22 substituting cycloheptyl amine for 4-ethyl aniline to provide the title compound (46%) as yellow color solid. ¹H NMR (300 MHz, DMSO-d₆) δ 1.61 (m, 6H), 1.79 (m, 2H), 1.96 (m, 2H), 2.06 (m, 2H), 3.00 (s, 6H), 4.81 (m, 1H), 8.23 (s, 1H), 8.73 (s, 1H), 8.91 (s, 1H); M/Z (DCI/NH₃) 343 (M+H)⁺. Anal. calcd for C₁₈H₂₂N₄OS: C, 63.13; H, 6.48; N, 16.36. Found: C, 62.93; H, 6.50; N, 16.16.

### Example 26

### 3-(4-ethylphenyl)-9-pyrrolidin-1-ylpyrido[4′,3′:4,5]thieno[3,2-d]pyrimidin-4(3H)-one

Compound was prepared using the procedure described in Example 22 substituting pyrrolidine in step 2 for dimethylamine to provide the title compound (34%) as yellow colored solid. ¹H NMR (300 MHz, DMSO-d₆) δ 1.24 (t, J=9 Hz, 3H), 1.98 (m, 4H), 2.69 (q, J=9 Hz, 2H), 3.63 (m, 4H), 7.42 (d, J=9 Hz, 2H), 7.48 (d, J=9 Hz, 2H), 8.18 (s, 1H), 8.59 (s, 1H), 8.78 (s, 1H); M/Z (DCI/NH₃) 377 (M+H)⁺. Anal. calcd for C₂₁H₂₀N₄OS: C, 67.00; H, 5.35; N, 14.88. Found: C, 66.77; H, 5.35; N, 14.44.

### Example 27

### 3-(3-fluoro-4-methylphenyl)-9-pyrrolidin-1-ylpyrido[4′,3′:4,5]thieno[3,2-d]pyrimidin-4(3H)-one

Compound was prepared using the procedure described in Example 22 substituting 3-fluoro-4-methyl aniline for 4-ethyl aniline to provide the title compound (31%) as yellow colored solid. ¹H NMR (300 MHz, DMSO-d₆) δ 1.98 (m, 4H), 2.38 (d, J=1.5 Hz, 3H), 3.64 (m, 4H), 7.38 (m, 1H), 7.52 (m, 2H), 8.17 (s, 1H), 8.60 (s, 1H), 8.78 (s, 1H); MS (DCI/NH₃) 381 (M+H)⁺. Anal. calcd for C₂₀H₁₇FN₄OS: C, 63.14; H, 4.50; N, 14.73. Found: C, 62.97; H, 4.74; N, 13.86.

### Example 28

### 3-(3-bromophenyl)-9-(dimethylamino)pyrido[3′,2′:4,5]thieno[3,2-d]pyrimidin-4(3H)-one

9.7 g of 4-Dimethylamino-3-(dimethylamino-methyleneamino)-thieno[2,3-b]pyridine-2-carboxylic acid methyl ester (see example 1 for procedure and alternative name), 8.4 g of 3-bromoaniline, and 250 mg of p-toluenesulfonic acid in 100 mls of toluene was heated to reflux at 130° C. for 24 hours. The solvent was removed by rotory evaporation, 20 mls of methanol were added and the reaction cooled to 4° C. The precipitate was filtered, washed with ice cold methanol and oven dried to yield 2.5 g of product. 1H NMR (300 MHz, DMSO-D6) δ ppm 3.12 (s, 6H) 6.94 (d, J=5.76 Hz, 1H) 7.56 (t, J=7.97 Hz, 1H) 7.60-7.68 (m, 1H) 7.74-7.79 (m, 1 H) 7.91 (t, J=1.86 Hz, 1 H) 8.39 (d, J=5.76 Hz, 1 H) 8.60 (s, 1 H). MS (DCI/NH3) m/z 402 (M+H)⁺.

### Example 29

### 3-(4-ethylphenyl)-9-(methylamino)pyrido[3′,2′:4,5]thieno[3,2-d]pyrimidin-4(3H)-one

To a solution of (A-794282) Example 16 (53 mg, 0.15 mmol) in formic acid (5 mL) at 0° C. was added 30% H₂O₂ (1 mL) and the mixture was allowed to warm to room temperature for 24 h. Solid NaHCO₃ was added to pH 8 and the mixture was extracted with ethyl acetate, washed with brine and dried with anhydrous MgSO₄. The ethyl acetate was removed under reduced pressure and the residue was chromatographed (silica gel, hexane-EtOAc 1:1) to provide 35 mg (70%) of the desired product and 10 mg (18%) of product Example 60. 1H NMR (300 MHz, DMSO-d₆) δ 1.24 (t, J=7 Hz, 3H), 2.70 (q, J=7 Hz, 2H), 3.03 (d, J=4.5 Hz, 3H), 6.66 (d, J=6 Hz, 1H), 7.41 (d, J=9 Hz, 2H), 7.48 (d, J=9 Hz, 2H), 7.71 (q, J=4.5 Hz, 1H), 8.31 (d, J=6 Hz, 1H), 8.59 (s, 1H); MS (DCI/NH3) m/z 337 (M+H)+. Analysis calcd for C18H16N4OS.0.25H2O: C, 63.42; H, 4.88; N, 16.43. Found: C, 63.18, H, 4.49; N, 16.36.

### Example 30

### 3-(4-methylphenyl)-9-pyrrolidin-1-ylpyrido[4′,3′:4,5]thieno[3,2-d]pyrimidin-4(3H)-one

Compound was prepared using the procedure described in Example 26 substituting p-toluidine for 4-ethyl aniline to provide the title compound (43%) as yellow colored solid. ¹H NMR (300 MHz, DMSO-d₆) δ 1.98 (m, 4H), 2.42 (s, 3H), 3.64 (m, 4H), 7.39 (d, J=9 Hz, 2H), 7.47 (d, J=9 Hz, 2H), 8.17 (s, 1H), 8.58 (s, 1H), 8.80 (s, 1H); MS (DCI/NH₃) m/z 363 (M+H)⁺. Anal. calcd for C₂₀H₁₈N₄OS: C, 66.28; H, 5.01; N, 15.46. Found: C, 66.01; H, 4.94; N, 15.76.

### Example 31

### 3-cycloheptyl-9-pyrrolidin-1-ylpyrido[4′,3′:4,5]thieno[3,2-d]pyrimidin-4(3H)-one

Compound was prepared using the procedure described in Example 26 substituting cycloheptyl amine for 4-ethyl aniline in the final step to provide the title compound (43%) as yellow colored solid. ¹H NMR (300 MHz, DMSO-d₆) δ 1.61 (m, 6H), 1.79 (m, 2H), 1.98 (m, 6H), 2.06 (m, 2H), 3.64 (m, 4H), 4.79 (m, 1H), 8.15 (s, 1H), 8.68 (s, 1H), 8.74 (s, 1H); MS (DCI/NH₃) m/z 369 (M+H)⁺. Anal. calcd for C₂₀H₂₄N₄OS: C, 65.19; H, 6.56; N, 15.20. Found: C, 65.01; H, 6.84; N, 15.56.

### Example 32

### 9-Dimethylamino-3-(2′-methyl-biphenyl-3-yl)-3H-pyrido[3′,2′:4,5]thieno[3,2-d]pyrimidin-4-one

110 mg of 3-(3-Bromo-phenyl)-9-dimethylamino-3H-pyrido[3′,2′:4,5]thieno[3,2-d]pyrimidin-4-one (Example 28) in 3 mls of dimethoxyethane was flushed with nitrogen. 35 mg of o-tolylboronic acid, 9 mg of tetrakis(triphenylphosphine)palladium(0), and 1 ml of 1M potassium carbonate are added and the reaction again flushed with nitrogen. The reaction mixture is heated in a sealed tube at 80C for 18 hours. The reaction is cooled, 20 mls of dichloromethane is added and the reaction extracted with saturated NH₄Cl, the organic phase is dried over MgSO₄ and the solvent evaporated. The product is isolated from the crude mixture by reverse phase HPLC using a gradient of H₂O/CH₃CN/TFA as the mobile phase to give 100 mg of product. 1H NMR (300 MHz, DMSO-D6) δ ppm 2.32 (s, 3H) 3.16 (s, 6H) 6.97 (d, J=6.10 Hz, 1H) 7.23-7.39 (m, 4H) 7.44-7.74 (m, 4H) 8.40 (d, J=5.76 Hz, 1H) 8.68 (s, 1H). MS (DCI/NH3) m/z 413 (M+H)⁺.

### Example 33

### 9-Dimethylamino-3-(2′-methoxy-biphenyl-3-yl)-3H-pyrido[3′,2′:4.5]thieno[3,2-d]pyrimidin-4-one

The compound was prepared using procedure described in Example 32 substituting 2-methoxyphenylboronic acid for o-tolylboronic acid. 1H NMR (300 MHz, DMSO-D6) δ ppm 3.17 (s, 6 H) 3.80 (s, 3 H) 6.98 (d, J=5.76 Hz, 1H) 7.06 (dt J=7.46, 1.02 Hz, 1H) 7.15 (dd, J=8.65, 0.85 Hz, 1 H) 7.35-7.43 (m, 2 H) 7.55 (dt, J=7.80, 1.86 Hz, 1 H) 7.58-7.65 (m, 1 H) 7.68 (d, J=1.70 Hz, 1 H) 7.68-7.71 (m, 1 H) 8.41 (d, J=6.10 Hz, 1 H) 8.67 (s, 1 H). MS (DCI/NH3) m/z 429 (M+H)⁺.

### Example 34

### 3-(2′-Chloro-biphenyl-3-yl)-9-dimethylamino-3H-pyrido[3′,2′:4,5]thieno[3,2-d]pyrimidin-4-one

The compound was prepared using the procedure described in Example 32 substituting 2-chlorophenylboronic acid for o-tolylboronic acid. 1H NMR (300 MHz, DMSO-D6) δ ppm 3.17 (s, 6 H) 6.98 (d, J=5.76 Hz, 1 H) 7.41-7.51 (m, 2 H) 7.51-7.56 (m, 1 H) 7.58-7.66 (m, 2 H) 7.66-7.69 (m, 2 H) 7.68-7.71 (m, 1 H) 8.41 (d, J=5.76 Hz, 1 H) 8.68 (s, 1 H) ESI m/z 433 (M+H)⁺.

### Example 35

### 3-(3-fluoro-4-methylphenyl)-9-pyrrolidin-1-ylpyrido[3′,2′:4.5]thieno[3,2-d]pyrimidin-4(3H)-one

The compound was prepared according to the procedure outlined in Example 40 pyrrolidine for diethylamine in step one and and substituting 3-fluoro-4-methyl aniline for 4-ethyl aniline in the final step; providing the title compound (46%) as off white color solid. ¹H NMR (300 MHz, DMSO-d₆) δ 1.95 (m, 4H), 2.33 (d, J=1.5 Hz, 3H), 3.74 (m, 4H), 6.75 (d, J=6 Hz, 1H), 7.39 (d, J=9 Hz, 2H), 7.44 (d, J=9 Hz, 2H), 8.23 (d, J=6 Hz, 1H), 8.66 (s, 1H); MS (DCI/NH₃) m/z 381 (M+H)⁺. Anal. calcd for C₂₀H₁₇FN₄OS: C, 63.14; H, 4.50; N, 14.73. Found: C, 62.87; H, 4.52; N, 14.47.

### Example 36

### 9-(dimethylamino)-3-(4-ethylphenyl)-2-methylpyrido[3′,2′:4,5]thieno[3,2-d]pyrimidin-4(3-one

### Example 36A

### 3-amino-4-(dimethylamino)thieno[2,3-b]pyridine-2-carboxylic acid

A suspension of 5-amino-4-dimethylaminothieno[8,9-b]pyridine-6-carboxylic acid methyl ester (1.25 g, 5.0 mmol) was suspended in 4% ethanolic NaOH (10 ml). The mixture was refluxed for one hour. Yellow solid precipitated out, filtered the solid, vacuum dried to obtain desired sodium salt (94%), which was carried to the next step.

### Example 36B

### 9-(dimethylamino)-2-methyl-4H-pyrido[3′,2′:4,5]thieno[3,2-d][1.3]oxazin-4-one

The sodium salt from step 1(1.05 g, 4.1 mmol) was suspended in acetic anhydride (6 ml). The mixture was refluxed for 4 hours, cooled to room temperature, concentrated, residue slurried in toluene (10 ml), filtered solid to obtain 0.95 g of desired 35a oxizinone (93%) as yellow color solid.

### Example 36

### 9-(dimethylamino)-3-(4-ethylphenyl)-2-methylpyrido[3′,2′:4,5]thieno[3,2-d]pyrimidin-4(3H)-one

A mixture of Example 36B (0.261 g, 1.0 mmol), 4-ethyl aniline (0.24 g, 2.0 mmol), and acetic acid (10 ml) was refluxed under N₂ for 4 hours. Reaction mixture was cooled to room temperature, concentrated under vacuum, residue purified by flash column chromatography (silica gel, 1:1. Hexane:EtOAc) to give 0.045 g (14%) of desired pyrimidone as beige color solid. 1H NMR (300 MHz, DMSO-d₆) δ 1.25 (t, J=9 Hz, 3H), 2.23 (s, 3H), 2.71 (q, J=9 Hz, 2H), 3.15 (s, 6H), 6.83 (d, J=6 Hz, 1H), 7.36 (d, J=9 Hz, 2H), 7.42 (d, J=9 Hz, 2H), 8.37 (d, J=6 Hz, 1H); MS (DCI/NH3) m/z 365 (M+H)+. Anal. calcd for C20H20N4OS: C, 65.91; H, 5.53; N, 15.37. Found: C, 65.54; H, 5.43; N, 14.96.

### Example 37

### 9-(dimethylamino)-2-methyl-3-(4-methylphenyl)pyrido[3′,2′:4,5]thieno[3,2-d]pyrimidin-4(3H)-one

Compound was prepared using procedure described in Example 36 substituting p-toluidine for 4-ethyl aniline to provide the title compound (28%) as beige color solid. ¹H NMR (300 MHz, DMSO-d₆) δ 2.24 (s, 3H), 2.42 (s, 3H), 3.13 (s, 6H), 6.90 (d, J=6 Hz, 1H), 7.44 (d, J=9 Hz, 2H), 7.49 (d, J=9 Hz, 2H), 8.35(d, J=6 Hz, 1H); MS (DCI/NH₃) m/z 351 (M+H)⁺. Anal. calcd for C₁₉H₁₈N₄OS: C, 65.12; H, 5.18; N, 15.99. Found: C, 64.95; H, 4.85; N, 15.70.

### Example 38

### 3-(4-methylphenyl)-9-pyrrolidin-1-ylpyrido[3′,2′:4,5]thieno[3,2-d]pyrimidin-4(3H)-one

The compound was prepared using the procedure described in Example 35 p-toluidine for 4-ethyl aniline; providing the title compound (49%) as off white color solid. ¹H NMR (300 MHz, DMSO-d₆) δ 1.94 (m, 4H), 2.42 (s, 3H), 3.74 (m, 4H), 6.75 (d, J=6 Hz, 1H), 7.38 (d, J=9 Hz, 2H), 7.44 (d, J=9 Hz, 2H), 8.23 (d, J=6 Hz, 1H), 8.50 (s, 1H); MS (DCI/NH₃) m/z 363 (M+H)⁺. Anal. calcd for C₂₀H₁₈N₄OS: C, 66.28; H, 5.01; N, 15.46. Found: C, 66.17; H, 4.90; N, 15.28.

### Example 39

### 9-(dimethylamino)-3-(4-methylcyclohexyl)pyrido[3′,2′:4,5]furo[3,2-d]pyrimidin-4(3H)-one

### Example 39A

### 5-Amino-4-dimethylaminofuro[8,9-b]pyridine-6-carboxylic acid methyl ester

To a solution of methyl glycolate (2.7 mL, 35 mmol) in anhydrous THF (100 mL), sodium hydride (60%, 2.8 g, 70 mmol) was added in small portions at 0° C. A solution of 4-Dimethylamino-2-chloro-3-cyanopyridine (5.0 g, 27.6 mmol) in THF (25 mL) was added. The reaction mixture was allowed to stir for 3 day at room temperature. The reaction mixture was then heated to reflux for 2 h. After cooled down to room temperature, the reaction mixture was quenched with saturated NH₄Cl. Organic solvent was removed under vacuum, and the aqueous layer was extracted with dichloromethane (500 mL). Organic layer was separated and washed with water and brine, then dried over Na₂SO₄. The solvent was concentrated, and purified via column chromatography (SiO2, ethyl acetate:hexanes=1:9 then 1:4) to give 4.9 g product (76%). 1H NMR (CDCl₃, 300 MHz): δ=8.22 ppm (d, J=5.8 Hz, 1H), 7.27 (d, J=5.8 Hz, 1H), 5.27 (s, br, 2H), 3.94 (s, 3H), 3.01 (s, 6H). MS (ESI, M+1): 236.0.

### Example 39B

### 4-Dimethylamino-5-(dimethylaminomethyleneamino)furo[8,9-b]pyridine-6-carboxylic acid methyl ester

5-Amino-4-dimethylaminofuro[8,9-b]pyridine-6-carboxylic acid methyl ester (2.0 g, 8.5 mmol) was dissolved in ethanol (12 mL) and N,N-dimethylformamide dimethyl acetal (6 mL), and heated to reflux for 4.5 h. Excess of solvent and reagent were removed to give a yellow solid product (2.5 g, 100%). 1H NMR (CDCl3, 300 MHz): δ=8.12 ppm (d, J=5.8 Hz, 1H), 7.68 (s, 1H), 6.44 (d, J=5.8 Hz, 1H), 3.87 (s, 3H), 3.16 (s, 6H), 3.14 (s, br, 3H), 3.09 (s, br, 3H). MS (ESI, M+1): 291.1.

### Example 39

### 9-Dimethylamino-3-(4′-methyl-1′-cyclohexyl)-3H-5-thia-1,3,6-triazafluoren-4-one

Dimethylamino-5-(dimethylaminomethyleneamino)furo[8,9-b]pyridine-6-carboxylic acid methyl ester (150 mg, 0.52 mmol), para-toluenesulfonic acid (10 mg, 0.05 mmol) and 4-methylcyclohexylamine (88 mg, 0.78 mmol) were placed in flask with tolune (10 mL) and then heated to 130 C. for over night. Cooled down to room temperature. Solvent was removed under vacuum, and the residue was purified via column chromatography (SiO2, ethyl acetate:hexanes=1:9 then 1:4) to give a pure product (75 mg, 44%). 1H NMR (CDCl3/MeOD, 300 MHz): δ=8.19 ppm (m, 1H), 8.16 (m, 1H), 6.50 (m, 5.8 Hz, 1H), 4.90 (m, 1H), 3.42 (m, 6H), 1.50-2.10 (m, 8H), 1.30 (m, 1H), 1.00 (m, 3H). MS (ESI, M+1): 327.1.

### Example 40

### 9-(diethylamino)-3-(3-fluoro-4-methylphenyl)pyrido[3′,2′:4,5]thieno[3,2-d]pyrimidin-4(3H)-one

### Example 40A

### 4-diethylamino-5-cyano-6-chloropyridine

The mixture of 4,6-dichloro-5-cyanopyridine (2.0 g, 11.5 mmol) dissolved in DMF (10 ml) was cooled to 0° C. To this mixture was added diethylamine (2.5 ml, 24 mmol). The mixture was allowed to come to room temperature and stirred at that temperature for 2 hours and then poured into ice water. The precipitate formed was filtered, vacuum dried to obtain 2.1 g (87%) of desired nitrile as beige color solid. ¹H NMR (300 MHz, DMSO-d₆) δ 1.10 (t, J=9 Hz, 6H), 3.59 (q, J=9 Hz, 4H), 6.84 (d, J=6 Hz, 1H), 7.98 (d, J=6 Hz, 1H); MS (DCI/NH₃) m/z 210(M+H)⁺.

### Example 40

The compound was prepared using the procedure described in Example 22, substituting in step 3,4-diethylamino-5-cyano-6-chloropyridine, prepared above in step 1) for 3-dimethylamino-4-cyano-5-chloropyridine and substituting in the final step 3-fluoro-4-methyl aniline for 4-ethyl aniline. Providing the title compound (31%) as off white color solid. ¹H NMR (300 MHz, DMSO-d₆) δ 1.11 (t, J=9 Hz, 6H), 2.34 (d, J=1.5 Hz, 3H), 3.60 (q, J=9 Hz, 4H), 6.97 (d, J=6 Hz, 1H), 7.36 (dd, J=9 Hz, 1.5 Hz, 1H), 7.48 (m, 2H), 8.40 (d, J=6 Hz, 1H), 8.58 (s, 1H); MS (DCI/NH₃) m/z 383 (M+H)⁺. Anal. calcd for C₂₀H₁₉FN₄OS: C, 62.81; H, 5.01; N, 14.65. Found: C, 62.71; H, 5.01; N, 14.46.

### Example 41

### 9-N-Azetidinyl-3-(4′-ethylphenyl)-3H-5-thia-1,3,6-triazafluoren-4-one

### Example 41A

### 2-(3-dimethylamino-1-ethoxyallylidene)malononitrile

2-(1-ethoxyethylidene)malono-nitrile (110.0 g, 808 mmol) was dissolved in N,N-dimethylformamide dimethyl acetal (94%, 230 mL, 1.62 mol), and the reaction mixture was heated to reflux at 100° C. for 1 hr. Cooled down to room temperature. Solid was collected, and washed with cold methanol to give an orange solid product. The mother liquor was concentrated, and the solid was collected again, and washed with cold methanol. This procedure was repeated couple of times, and all the solid products were combined. The final residue was purified by a short column chromatography (SiO₂, ethyl acetate). The combined yield of the reaction is 78% (120.0 g) as a mixture of 2-(3-dimethylamino-1-ethoxy-allylidene)malononitrile (73.5%) and 2-(3-dimethylamino-1-methoxyallylidene)malono-nitrile (26.5%).

2-(3-dimethylamino-1-ethoxyallylidene)malononitrile: ¹H NMR (CDCl₃, 300 MHz): δ=7.47 ppm (d, J=12.6 Hz, 1H); 5.16 (d, J=12.6 Hz, 1H); 4.43 (q, J=7.1 Hz, 2H), 3.19 (s, br. 3H), 2.94 (s, br. 3H), 1.41 (t, J=7.1 Hz, 3H). MS (ESI, M+1): 192.1.

2-(3-dimethylamino-1-methoxyallylidene)malononitrile: ¹H NMR (CDCl₃, 300 MHz): δ=7.51 ppm (d, J=12.9 Hz, 1H); 5.10 (d, J=12.9 Hz, 1H); 4.11 (s, 3H), 3.19 (s, br. 3H), 2.94 (s, br. 3H).

MS (ESI, M+1): 178.0.

### Example 41B

### 2-Chloro-3-cyano-4-ethoxypyridine

To a slurry of a mixture of 2-(3-dimethylamino-1-ethoxy-allylidene)malononitrile and 2-(3-dimethylamino-1-methoxyallylidene)malono-nitrile (85.0 g, 445 mmol) from step 1, in methanol (1200 mL), HCl gas was introduced gently at 0° C. The reaction mixture became homogeneous in about 1 hr, and was allowed to stir under constant HCl flow for additional 14.5 hours at 0° C. N₂ was bubled though the reaction mixture for over night, and all the solvent was removed. The residue solid was re-dissolved in CH₂Cl₂, and washed with water/K₂CO₃/water. Organic layer was separated and dried over Na₂SO₄. Removal of salt and solvent gave a pure product (113.0 g, 98%). 1H NMR (CDCl₃, 300 MHz): δ=7.96 ppm (d, J=6.4 Hz, 1H); 6.59 (d, J=6.4 Hz, 1H); 3.30 (s, 6H). MS (ESI, M+1): 181.9.

### Example 41C

### 2-Bromo-3-cyano-4-hydroxypyridine

2-Chloro-3-cyano-4-ethoxypyridine (8.5 g, 46.7 mmol) from step 2, and HBr in acetic acid (30%, 85 mL) was heated to 100 C. for 2 hours. Cooled down to room temperature, solid was collected, and washed with cold water and dried under vacuum to give a white solid with 91.6% bromonation product, and 8.4% chlorination product.

2-Bromo-3-cyano-4-hydroxypyridine: ¹H NMR (DMSO-d₆, 300 MHz): δ=8.20 ppm (d, J=5.9 Hz, 1H); 6.95 (d, J=5.9 Hz, 1H). MS (ESI, M+1): 200.9.

2-Chloro-3-cyano-4-hydroxypyridine: ¹H NMR (DMSO-d₆, 300 MHz): δ=8.27 ppm (d, J=6.3 Hz, 1H); 6.98 (d, J=6.3 Hz, 1H). MS (ESI, M+1): 172.0.

### Example 41D

### 2-Bromo-3-cyano-4-(4′-methoxybenzyloxy)pyridine

A mixture of 2-Bromo-3-cyano-4-hydroxypyridine and 2-chloro-3-cyano-4-hydroxypyridine (10.0 g, 35.7 mmol) from step 3, was dissolved in DMF (50 mL), followed by NaH in portions (60%, 2.86 g, 71.5 mmol). 4-Methoxybenxyl chloride (6.85 mL, 50.3 mmol) was added. The reaction mixture was then heated to 60 C. for 2.5 hours and quenched with NH4Cl (saturated). The mixture was then extracted with ethyl acetate (5×20 mL). The combined organic layers were washed with water, and dried over Na2SO4. Column chromatographic purification (SiO2, ethyl acetate:hexanes=1:9) gave an off-white solid product (4.21 g, 37%) with 92% the title product and 8% of 2-chloro-3-cyano-4-(4′-methoxybenzyloxypyridine. 2-Bromo-3-cyano-4-(4′-methoxybenxyloxy)pyridine: ¹H NMR (CDCl₃, 300 MHz): δ=8.33 ppm (d, J=5.7 Hz, 1H); 7.34 (d, J=8.8 Hz, 2H), 6.94 (d, J=8.8 Hz, 2H), 5.23 (s, 2H), 3.83 (s, 3H). MS (ESI, M+1): 320.0.

2-Chloro-3-cyano-4-(4′-methoxybenyloxy)pyridine: ¹H NMR (CDCl₃, 300 MHz): δ=8.36 ppm (d, J=5.8 Hz, 1H); 7.31 (d, J=8.8 Hz, 2H), 6.89 (d, J=8.8 Hz, 2H), 4.63 (s, 2H), 3.82 (s, 3H). MS (ESI, M+1): 275.0.

### Example 41E

### 5-Amino-4-(4′-methoxybenyloxy)thieno[8,9-b]pyridine-6-carboxylic acid methyl ester

2-Bromo-3-cyano-4-(4′-methoxybenzyloxy)pyridine (4.0 g, 12.5 mmol) from step 4, was dissolved in DMF (20 mL), followed by methyl thioglycolate (1.2 mL, 13.2 mmol), and then sodium methoxide (95%, 1.56 g, 27.4 mmol) in portions at room temperature. The reaction mixture was allowed to stir over night, and then quenched with water. Solid was collected and washed with water several times, then dried under vacuum to give a almost pure product (3.7 g, 86%). 1H NMR (CDCl3, 300 MHz): δ=8.48 ppm (d, J=5.8 Hz, 1H), 7.39 (m, 2H), 6.96 (m, 2H), 6.76 (d, J=5.8 Hz, 1H), 6.53 (s, br. 1H), 5.19 (s, 2H), 3.85 (s, 3H), 3.84 (s, 3H). MS (ESI, M+1): 344.9.

### Example 41F

### 4-(4′-Methoxybenyloxy)-5-(dimethylaminomethyleneamino)thieno[8,9-b]pyridine-6-carboxylic acid methyl ester

5-Amino-4-(4′-methoxybenyloxy)thieno[8,9-b]pyridine-6-carboxylic acid methyl ester (3.7 g, 10.8 mmol) from step 5, was dissolved in N,N-dimethylformamide dimethyl acetal (30 mL), and heated to reflux for 10 hours. Excess of reagent was removed to give a yellow solid product (4.3 g, 100%). 1H NMR (CDCl3, 300 MHz): δ=8.47 ppm (d, J=5.8 Hz, 1H), 7.44 (s, 1H), 7.40 (m, 2H), 6.91 (m, 2H), 6.75 (d, J=5.8 Hz, 1H), 5.09 (s, 2H), 3.84 (s, 3H), 3.82 (s, 3H), 2.83 (s, br, 6H). MS (ESI, M+1): 400.0.

### Example 41G

### 9-(4′-Methoxybenyloxy)-3-(4′-ethylphenyl)-3H-5-thia-13,6-triazafluoren-4-one

4-(4′-Mwthoxybenyloxy)-5-(dimethylaminomethyleneamino)thieno[8,9-b]pyridine-6-car-boxylic acid methyl ester (4.0 g, 10.0 mmol) from step 6,4-ethylanaline (2.42 g, 20.0 mmol) and catalytic amount of p-toluenesulfonic acid (200 mg, 1.1 mmol) in toluene (50 mL) was heated under microwave to 160 C. for 1 h. The reaction mixture was then coolded and solvent was removed. The residue was purified via column chromatography (SiO2, ethyl acetate:hexanes=1:4, then 1:1) to give a solid product, which was recrystalized from cold methanol (1.733 g, 39%). 1H NMR (DMSO-d₆, 300 MHz): δ=8.64 ppm (d, J=5.5 Hz, 1H), 8.56 (s, 1H), 7.53 (d, J=8.6 Hz, 2H), 7.48 (d, J=8.3 Hz, 2H), 7.40 (d, J=8.3 Hz, 2H), 7.33 (d, J=5.5 Hz, 1H), 6.97 (d, J=8.6 Hz, 2H), 5.45 (s, 2H), 3.75 (s, 3H), 3.71 (q, J=7.4 Hz, 2H), 1.25 (t, J=7.4 Hz, 3H). MS (ESI, M+1): 444.0.

### Example 41H

### 9-Hydroxy-3-(4′-ethylphenyl)-3H-5-thia-1,36-triazafluoren-4-one

9-(4′-Methoxy-benyloxy)-3-(4′-ethylphenyl)-3H-5-thia-1,3,6-triazafluoren-4-one (1.6 g, 3.6 mmol) from step 7, was added to cold (0 C.) trifluoroactic acid (10 mL) in portions, and then allowed to stir for 1.2 h. Excess of TFA was removed under vacuum. The residue was allowed to sit over night, then treated with ethyl acetate (20 mL). Ethyl acetate was removed and the procedure was repeated several times till a yellow solid was obtained. Hexanes was added to the solid, and the mixture was sonicated. Solid was then collected to give a pure product (1.61 g, 100%). 1H NMR (DMSO-d6, 300 MHz): δ=8.54 ppm (s, 1H), 8.42 (d, J=5.9 Hz, 1H), 7.48 (d, J=6.6 Hz, 2H), 7.43 (d, J=6,6 Hz, 2H), 6.92 (d, J=5.9 Hz, 2H), 2.72 (q, J=7.5 Hz, 2H), 1.25 (t, J=7.5 Hz, 3H). MS (ESI, M+1): 324.0.

### Example 41I

### 9-(Triflurometanesulfonyl)-3-(4′-ethylphenyl)-3H-5-thia-1,3,6-triazafluoren-4-one

4-Hydroxy-7-(4′-ethylphenyl)-7H-9-thia-1,5,7-triazafluoren-8-one (1.66 g, 3.82 mmol) from step 8, N-phenyltrifluoromethanesulfonimide (3.42 g, 9.57 mmol) and N-ethyl-diisopropylamine (1.7 mL, 9.76 mmol) were dissolved in 1,4-dioxane (80 mL) at room temperature. The reaction mixture was allowed to stir for 3 days. Solvent was removed, and the residue was purified via column chromatography (SiO2, ethyl acetate:hexanes=1:9) to give a white solid product (1.42 g, 82%). ¹H NMR (CDCl3, 300 MHz): δ=8.88 ppm (d, J=5.1 Hz, 1H), 8.35 (s, 1H), 7.39 (m, 5H), 2.74 (q, J=7.5 Hz, 2H), 1.31 (t, J=7.5 Hz, 3H). MS (ESI, M+1): 455.9

### Example 41

### 9-N-Azetidinyl-3-(4′-ethylphenyl)-3H-5-thia-1,3,6-triazafluoren-4-one

9-(Trifluromethanesulfonyl)-3-(4′-ethylphenyl)-3H-5-thia-1,3,6-triazafluoren-4-one (60 mg, 0.13 mmol) was dissolved in dichloromethane (2 mL). Azetidine hydrochloride (50 mg, 0.52 mmol) was neutralized with sodium hydroxide (1N, 1 mL, 1.0 mmol) in dichloromethane. Organic layer was separated and dried over sodium sulfate. The solution of azetidine in dichloromethane was then filtrated and added to the reaction mixture. The reaction mixture was then allowed to stir at room temperature for 1 day. Solvent was removed, and the crude mixture was purified via a short column chromatography (SiO₂, ethyl acetate:hexanes=1:4) to give a white solid product (42 mg, 89%). 1H NMR (CDCl3, 300 MHz): δ=8.26 ppm (d, J=6.1 Hz, 1H), 8.17 (s, 1H), 7.37 (m, 4H), 6.25 (d, J=6.1 Hz, 2H), 4.54 (m, 4H), 2.75 (q, J=7.8 Hz, 2H), 2.47 (m, 2H), 1.30 (t, J=7.8 Hz, 3H). MS (ESI, M+1): 363.0.

### Example 42

### 9-(N N-Ethylmethylamino)-3-(4′-ethylphenyl)-3H-5-thia-1,3,6-triazafluoren-4-one

Compound was prepared by procedure described for Example 41 substituting azetidine with ethylmethylamine hydrochloride. ¹H NMR (CDCl₃, 300 MHz): δ=8.43 ppm (d, J=6.6 Hz, 1H), 8.27 (s, 1H), 7.40 (d, J=8.4 Hz, 2H), 7.35 (d, J=8.4 Hz, 2H), 6.84 (d, J=6.6 Hz, 1H), 3.82 (q, J=7.2 Hz, 2H), 3.33 (s, 3H), 2.75 (q, J=7.8 Hz, 2H), 1.38 (t, 7.2 Hz, 3H), 1.30 (t, J=7.8 Hz, 3H). MS (ESI, M+1): 365.0.

### Example 43

### 9-(2′-Methoxyethylmethylamino)-3-(4′-ethylphenyl)-3H-5-thia-1,3,6-triazafluoren-4-one

Compound was prepared by procedure described for Example 41 substituting azetidine with 2-methoxyethylmethylamine. ¹H NMR (CDCl₃, 300 MHz): δ=8.42 ppm (d, J=5.8 Hz, 1H), 8.26 (s, 1H), 7.37 (m, 4H), 6.85 (d, J=5.8 Hz, 1H), 3.91 (t, J=5.4 Hz, 2H), 3.68 (t, J=5.4 Hz, 2H), 3.27 (s, 3H), 3.21 (s, 3H), 2.75 (q, J=7.8 Hz, 2H), 1.30 (t, J=7.8 Hz, 3H). MS (ESI, M+1): 395.0.

### Example 44

### 9-(2′-Dimethylaminoethylmethylamino)-3-(4′-ethylphenyl)-3H-5-thia-1,3,6-triaza-fluoren-4-one

Compound was prepared by procedure described for Example 41 substituting azetidine with 2-dimethylaminoethylmethylamine. ¹H NMR (CDCl₃, 300 MHz): δ=8.50 ppm (m, 1H), 8.34 (s, 1H), 7.40 (m, 4H), 7.0 (m, 1H), 4.26 (m, 2H), 3.59 (m, 2H), 3.32 (s, 3H), 2.92 (s, 6H), 2.75 (q, J=7.5 Hz, 2H), 1.30 (t, J=7.5 Hz, 3H). MS (ESI, M+1): 408.0.

### Example 45

### 9-[2′-(2″-Pyridinylethylmethylamino)]-3-(4′-ethylphenyl)-3H-5-thia-1,3,6-triaza-fluoren-4-one

Compound was prepared by procedure described for Example 41 substituting azetidine with 2-(2′-pyridinyl)ethylmethylamine. ¹H NMR (CDCl₃, 300 MHz): δ=8.67 ppm (d, J=8.3 Hz, 1H), 8.36 (d, J=6.6 Hz, 1H), 8.30 (s, 1H), 8.15 (t, J=6.9 Hz, 1H), 7.67 (t, J=7.2 Hz, 1H), 7.60 (d, J=7.5 Hz, 1H), 7.39 (d, J=8.4 Hz, 2H), 7.35 (d, J=8.4 Hz, 2H), 6.97 (d, J=6.6 Hz, 1H), 4.47 (t, J=6.6 Hz, 2H), 3.60 (t, J=6.6 Hz, 2H), 3.45 (s, 3H), 2.92 (s, 6H), 2.75 (q, J=7.8 Hz, 2H), 1.30 (t, J=7.8 Hz, 3H). MS (ESI, M+1): 442.0.

### Example 46

### 9-(2′,2′,2′-Trifluoroethylamino)-3-(4′-ethylphenyl)-3H-5-thia-1,3,6-triazafluoren-4-one

Compound was prepared by procedure described for Example 41 substituting azetidine with 2,2,2-trifluorethylamine. ¹H NMR (CDCl₃, 300 MHz): δ=8.94 ppm (t, J=6.9 Hz, 1H), 8.55 (d, J=6.5 Hz, 1H), 8.32 (s, 1H), 7.97 (s, br. 1H), 7.40 (d, J=8.4 Hz, 2H), 7.34 (d, J=8.4 Hz, 2H), 6.82 (d, J=6.5 Hz, 1H), 4.16 (m, 2H), 2.76 (q, J=7.5 Hz, 2H), 1.31 (t, J=7.5 Hz, 3H). MS (ESI, M+1): 404.9.

### Example 47

### 9-(1′-Cyanomethylamino)-3-(4′-ethylphenyl)-3H-5-thia-1,3,6-triaza-fluoren-4-one

Compound was prepared by procedure described for Example 41 substituting azetidine with cyanomethylamine. ¹H NMR (CDCl₃, 300 MHz): 3=8.71 ppm (t, J=5.9 Hz, 1H), 8.62 (d, J=6.5 Hz, 1H), 8.29 (s, 1H), 7.41 (d, J=8.4 Hz, 2H), 7.35 (d, J=8.4 Hz, 2H), 6.79 (d, J=6.5 Hz, 1H), 4.47 (d, J=5.9 Hz, 2H), 2.76 (q, J=7.5 Hz, 2H), 1.31 (t, J=7.5 Hz, 3H). MS (ESI, M+1): 361.9.

### Example 48

### 9-Cyclopropylamino-3-(4′-ethylphenyl)-3H-5-thia-1,3,6-triazafluoren-4-one

Compound was prepared by procedure described for Example 41 substituting azetidine with cyclopropylamine. ¹H NMR (CDCl₃, 300 MHz): δ=8.67 ppm (s, 1H), 8.49 (d, J=6.6 Hz, 1H), 8.27 (s, 1H), 7.40 (d, J=8.0 Hz, 2H), 7.34 (d, J=8.0 Hz, 2H), 7.05 (d, J=6.6 Hz, 1H), 2.80 (m. 1H), 2.76 (q, J=7.8 Hz, 2H), 1.31 (t, J=7.8 Hz, 3H), 1.09 (m, 2H), 0.83 (m, 2H). MS (ESI, M+1): 363.1.

### Example 49

### 9-(diethylamino)-3-(4-methylphenyl)pyrido[3′,2′:4,5]thieno[3,2-d]pyrimidin-4(3H)-one

The compound was prepared following the procedure described in Example 40 substituting p-toluidine for 4-ethyl aniline to provide the title compound (53%) as off white color solid. ¹H NMR (300 MHz, DMSO-d₆) δ 1.13 (t, J=9 Hz, 6H), 2.41 (s, 3H), 3.60 (q, J=9 Hz, 4H), 6.97 (d, J=6 Hz, 1H), 7.39 (d, J=9 Hz, 2H), 7.44(d, J=9 Hz, 2H), 8.39 (d, J=6 Hz, 1H), 8.56 (s, 1H); MS (DCI/NH₃) m/z 365 (M+H)⁺. Anal. calcd for C₂₀H₂₀N₄OS.0.25H₂O: C, 65.38; H, 5.57; N, 14.86. Found: C, 65.05; H, 5.49; N, 15.19.

### Example 50

### 3-cycloheptyl-9-(diethylamino)pyrido[3′,2′:4,5]thieno[3,2-d]pyrimidin-4(3H)-one

The compound was prepared following the procedure described in Example 40 substituting 4-cycloheptyl amine for 4-ethyl aniline to provide the title compound (33%) as off white color solid. ¹H NMR (300 MHz, DMSO-d₆) δ 1.12 (t, J=9 Hz, 6H), 1.61 (m, 6H), 1.79 (m, 2H), 1.98 (m, 2H), 2.06 (m, 2H), 3.56 (q, J=9 Hz, 4H), 4.79 (m, 1H), 6.95 (d, J=6 Hz, 1H), 8.36 (d, J=6 Hz, 1H), 8.66 (s, 1H); MS (DCI/NH₃) m/z 371 (M+H)⁺. Anal. calcd for C₂₀H₂₆N₄OS: C, 64.83; H, 7.07; N, 15.12. Found: C, 64.59; H, 7.07; N, 14.76.

### Example 51

### 9-Dimethylamino-3-(2′-hydroxy-biphenyl-3-yl)-3H-pyrido[3′,2′:4,5]thieno[3,2-d]pyrimidin-4 one

Compound prepared using procedure described in Example 32 substituting 2-hydroxyphenylboronic acid for o-tolylboronic acid. 1H NMR (300 MHz, DMSO-D6) δ ppm 3.13 (s, 6H) 6.86-7.01 (m, 3H) 7.15-7.25 (m, 1H) 7.37 (dd, J=7.80, 1.70 Hz, 1H) 7.49-7.55 (m, 1H) 7.61 (t, J=7.80 Hz, 2H) 7.71-7.79 (m, 2H) 8.39 (d, J=5.76 Hz, 1H) 8.65 (s, 1H). ESI m/z 415 (M+H)⁺.

### Example 52

### 3-(9-Dimethylamino-4-oxo-4H-pyrido[3′,2′:4,5]thieno[3,2-d]pyrimidin-3-yl)-benzonitrile

Compound prepared using procedure described in Example 28 substituting 3-cyanoaniline for 3-bromoanaline. 1H NMR (300 MHz, DMSO-D6) δ ppm 3.13 (s, 6 H) 6.95 (d, J=5.76 Hz, 1 H) 7.81 (t, J=7.97 Hz, 1 H) 8.00 (ddd, J=8.14, 2.03, 1.02 Hz, 1 H) 8.04 (dt, J=7.80, 1.19 Hz, 1 H) 8.19 (t, J=1.86 Hz, 1 H) 8.40 (d, J=5.76 Hz, 1 H) 8.65 (s, 1 H) ESI m/z 348 (M+H)⁺.

### Example 53

### 9-Dimethylamino-3-(3-thiophen-3-yl-phenyl)-3H-pyrido[3′,2′:4,5]thieno[3,2-d]pyrimidin-4-one

Compound prepared using procedure described in Example 32 substituting 3-thiopheneboronic acid for o-tolylboronic acid. 1H NMR (300 MHz, DMSO-D6) d ppm 3.13 (s, 6 H) 6.94 (d, J=5.76 Hz, 1 H) 7.51 (ddd, J=7.80, 2.03, 1.02 Hz, 1 H) 7.59-7.71 (m, 3 H) 7.88-7.94 (m, J=7.80 Hz, 1 H) 7.97 (t, J=1.86 Hz, 1 H) 8.02 (dd, J=2.88, 1.53 Hz, 1 H) 8.40 (d, J=5.76 Hz, 1 H) 8.66 (s, 1 H) ESI m/z 405 (M+H)⁺.

### Example 54

### 3-(9-Dimethylamino-4-oxo-4H-pyrido[32′:4,5]thieno[3,2-d]pyrimidin-3-yl)-N-hydroxy-benzamidine

### Example 54A

### 3-[9-(dimethylamino)-4-oxopyrido[3′,2′:4,5]thieno[3,2-d]pyrimidin-3(4H)-yl]-N′-hydroxybenzenecarboximidamide

0.22 g of 3-(9-Dimethylamino-4-oxo-4H-pyrido[3′,2′:4,5]thieno[3,2-d]pyrimidin-3-yl)-benzonitrile (Example 52), 0.13 g of Hydroxylamine hydrochloride, 0.26 mls of triethylamine in 5 mls of ethanol is heated in a sealed tube at 60C for 18 hours. The solvent is removed and the product purified by flash chromatography using methanol and dichloromethane. ESI m/z 381 (M+H)⁺.

### Example 54B

### 9-Dimethylamino-3-[3-(5-methyl-[I 12,4]oxadiazol-3-yl)-phenyl]-3H-pyrido[3′,2′:4,5]thieno[3,2-d]pyrimidin-4-one

0.13 g of Example 54A, 0.35 mls of acetic anhydride in 2 mls of pyridine is allowed to react at room temperature for 30 minutes, 0.2 g of potassium carbonate is added and the reaction is heated in a sealed tube at 90C for 30 minutes. The reaction is filtered and the solvent evaporated, final product is purified by flash chromatography using methanol and dichloromethane. 1H NMR (300 MHz, DMSO-D6) δ ppm 2.69 (s, 3 H) 3.13 (s, 6 H) 6.94 (d, J=5.76 Hz, 1 H) 7.74-7.88 (m, 2 H) 8.16 (dt, J=6.87, 1.82 Hz, 1 H) 8.20-8.24 (m, 1 H) 8.40 (d, J=5.76 Hz, 1 H) 8.66 (s, 1 H). ESI m/z 405 (M+H)⁺.

### Example 55

### 9-Dimethylamino-3-[3-(5-phenyl-[1,2,4]oxadiazol-3-yl)-phenyl]-3H-pyrido[3′,2′:4,5]thieno[3,2-d]pyrimidin-4-one

0.07 g of 3-(9-Dimethylamino-4-oxo-4H-pyrido[3′,2′:4,5]thieno[3,2-d]pyrimidin-3-yl)-N-hydroxy-benzamidine (Example 54, step 1), 0.02 mls of benzoyl chloride, and 0.2 g of potassium carbonate in 2 mls of pyridine are heated in a sealed tube at 90 C. for 18 hours. The reaction is filtered and the solvent removed, final product is obtained by flash chromatography using hexane and ethyl acetate. 1H NMR (300 MHz, DMSO-D6) δ ppm 3.14 (s, 6H) 6.95 (d, J=5.76 Hz, 1 H) 7.63-7.80 (m, 3 H) 7.81-7.91 (m, 2 H) 8.19-8.25 (m, 2 H) 8.28 (dt, J=6.61, 2.03, 1.86 Hz, 1 H) 8.33 (t, J=1.86 Hz, 1 H) 8.40 (d, J=5.76 Hz, 1 H) 8.69 (s, 1 H). ESI m/z 467 (M+H)⁺.

### Example 56

### 9-(dimethylamino)-3-(4-ethylphenyl)pyrido[3′,2′:4,5]thieno[3,2-d]pyrimidin-4(3H)-one 6-oxide

A mixture of (A-794282) Example 16 (175 mg, 0.5 mmol), MeReO₃ (MTO) (0.7 mg, 0.0025 mmol) and 30% H₂O₂ (0.1 mL, 1 mmol) in CH₂Cl₂ (10 mL) was stirred at room temperature for 20 h. After added MnO2 (10 mg) and the mixture was stirred for additional 30 min. The mixture was then concentrated under reduced pressure, added AcOEt and the acetate solution washed with brine, dried with anhydrous MgSO4 and concentrated under reduced pressure. The residue was purified by column chromatography to afford 12 mg of the desired product. ¹H NMR (300 MHz, CDCl₃) δ 1.30 (t, J=7 hZ, 3H), 2.75 (q, J=7 Hz, 2H), 3.18 (s, 6H), 6.82 (d, J=6 Hz, 1H), 7.38 (2d, J=9 Hz, 4H), 8.30 (d, J=6 Hz, 1H), 8.33 (s, 1H); MS (DCI/NH₃) m/z 367 (M+H)⁺.

### Example 57

### 9-Dimethylamino-3-(3-{1-[(E)-methoxyimino]-ethyl}-phenyl)-3H-pyrido[3′,2′:4,5]thieno[3,2-d]pyrimidin-4-one

### Example 57A

### 3-(3-acelylphenyl)-9-(dimethylamino)pyrido[3′,2′:4,5]thieno[3,2-d]pyrimidin-4(3H)-one

Compound prepared using procedure described in Example 28 substituting 3′-aminoacetophenone for 3-bromoanaline. ESI m/z 365 (M+H)⁺.

### Example 57

### 9-Dimethylamino-3-(3-{1-[(E)-methoxyimino]-ethyl}-phenyl)-3H-pyrido[3′,2′:4,5]thieno[3,2-d]pyrimidin-4-one

0.3 g of 3-(3-Acetyl-phenyl)-9-dimethylamino-3H-pyrido[3′,2′:4,5]thieno[3,2-d]pyrimidin-4-one prepared in step 1, and 0.36 g of methoxyl amine hydrochloride in 5 mls of pyridine are heated in a sealed tube at 60C for 18 hours. The reaction is cooled diluted with 20 mls of water and the precipitate filtered, resolubilized in dichloromethane dried with MgSO₄ Final product purified by flash chromatography using dichloromethane and ethyl acetate. 1H NMR (300 MHz, DMSO-D6) d ppm 2.23 (s, 3 H) 3.13 (s, 6 H) 3.94 (s, 3 H) 6.94 (d, J=5.76 Hz, 1H) 7.61-7.64 (m, 2 H) 7.83 (ddd, J=5.00, 3.81, 1.70 Hz, 1 H) 7.87 (ddd, J=1.95, 1.36, 1.10 Hz, 1 H) 8.39 (d, J=5.76 Hz, 1 H) 8.61 (s, 1 H). ESI m/z 394 (M+H)⁺.

### Example 58

### 9-Dimethylamino-3-(3-{1-hydroxyimino-ethyl}-phenyl)-3H-pyrido[3′,2′:4.5]thieno[3,2-d]pyrimidin-4-one

Compound prepared as described in example 57 substituting hydroxylamine hydrochloride for methoxylamine hydrochloride. 1H NMR (300 MHz, DMSO-D6) δ ppm 2.20 (s, 3 H) 3.13 (s, 6 H) 6.95 (d, J=5.76 Hz, 1 H) 7.55-7.63 (m, 2 H) 7.79-7.86 (m, 2 H) 8.40 (d, J=5.76 Hz, 1 H) 8.62 (s, 1 H) 11.38 (s, 1 H). ESI m/z 397.7 (M+H)⁺.

### Example 59

### 8-Chloro-9-dimethylamino-3-(4-ethylphenyl)-3H-5-thia-1,3,6-triazafluoren-4-one

9-Dimethylamino-3-(4-ethylphenyl)-3H-5-thia-1,3,6-triazafluoren-4-one (Example 16) (350 mg, 1.0 mmol) was dissolved in acetonitrle (10 mL), followed by N-chlorosuccinimide (300 mg, 2.2 mmol) at room temperature. The reaction mixture was allowed to stir for 1 d. at room temperature. Solvent was removed. The residue was then purified via column chromatography (SiO2, ethyl acetate:hexanes=1:9) to give a pure product (65 mg, 17%). ¹H NMR (CDCl3/MeOD, 300 MHz): δ=8.49 ppm (s, 1H), 8.27 (s, 1H), 7.38 (m, 4H), 3.28 (s, 6H), 2.76 (q, J=7.5 Hz, 2H), 1.30 (t, 7.5 Hz, 3H). MS (ESI, M+1): 384.9.

### Example 60

### 3-(4-acetylphenyl)-9-(methylamino)pyrido[3′,2′:4,5]thieno[3,2-d]pyrimidin-4(3H)-one

The compound was produced and isolated (10 mg, 18%) from reaction described for Example 28. 1H NMR (300 MHz, DMSO-d₆) δ 2.65 (s, 3H), 3.04 (d, J=4.5 Hz, 3H), 6.67 9d, J=6 Hz, 1H), 7.72 (q, J=4.5 Hz, 1H), 7.78 (d, J=9 Hz, 2H), 8.25 (d, J=9 Hz, 2H), 8.32 (d, J=6 Hz, 1H), 8.65 (s, 1H); MS (DCI/NH3) m/z 351 (M+H)+.

### Example 61

### 2-butyl-9-(dimethylamino)-3-(4-ethylphenyl)pyrido[3′,2′:4,5]thieno[3,2-d]pyrimidin-4(3H)-one

The compound was prepared according to the procedure outlined in Example 36, substituting valeric anhydride for acetic anhydride in step 1. ¹H NMR(300 MHz, CDCL₃) δ: 0.85 (t, J=11.20 Hz, 2H), 1.22-1.38 (m, 6H), 1.70-1.82 (m, 2H), 2.55 (t, J=11.87 Hz, 2H), 2.75 (t, J=15.26 Hz, 2H), 3.21 (s, 6H), 6.77 (d, J=5.76 Hz, 1H), 7.18 (d, J=8.48 Hz, 2H), 7.39 (d, 8.48 Hz, 2H), 8.40 (d, J=5.76 Hz, 1H). m/e DCI/NH₃ m/z 407 (M+H)⁺; Calc for C₂₃H₂₆N₄SO: C, 67.95; H, 6.45; N, 13.78. Found: C, 68.05; H, 6.16; N, 13.51.

### Example 62

### 9-(dimethylnitroryl)-3-(4-ethylphenyl)-4a,9b-dihydropyrido[3′,2′:4,5]thieno[3,2-d]pyrimidin-4(3H)-one

Oxidation was conducted according to the procedure of L. Kaczmarek, R. Balicki, P. Nantka-Namirski, Chem. Ber., 125, 1965-1966, 1992. Hydrogen peroxide-urea complex (UHP) (1.7 g, 9 mmol) was added to a phthalic anhydride (0.88 g, 6 mmol) in methylene chloride (50 mL). After 5 minutes, 9-(dimethylamino)-3-(4-ethylphenyl)-4a,9b-dihydropyrido[3′,2′:4,5]thieno[3,2-d]pyrimidin-4(3H)-one (1.0 g, 2.86 mmol) was added and the mixture was stirred until consumption of starting material (−30 minutes). A 2N solution of Na₂CO₃ (10 mL) was added and the layers were separated. The water layer was extracted twice with methylene chloride and the combined organics were dried with anhydrous MgSO₄, filtered and concentrated under reduced pressure. The residue was purified by column chromatography (CH₂Cl₂-EtOAc 9:1) to afford the desired product. ¹H NMR (300 MHz, DMSO-d₆) δ 1.23 (t, J=7 Hz, 3H), 2.71 (q, J=7 Hz, 2H), 2.91 (s, 6H), 7.42 (d, J=9 Hz, 2H), 7.48 (d, J=9 Hz, 2H), 7.55 (d, J=6 Hz, 1H), 8.58 (s, 1H), 8.65 (d, J=6 Hz, 1H). MS (DCI/NH₃) m/z 367 (M+H)⁺. Analysis calculated for C₁₉H18N₄O₂S.0.2H₂O: C, 61.67; H, 5.01; N, 15.14. Found: C, 61.56; H, 4.77; N, 14.98.

### Example 63

### 3-azepan-1yl-9-(dimethylnitroryl)-4a,9b-dihydropyrido[3′,2′:4.5]thieno[3,2-d]pyrimidin-4(3H)-one

The desired product was prepared according to the procedure outlined in Example 62 substituting 3-azepan-1-yl-9-(dimethylamino)-4a,9b-dihydropyrido[3′,2′:4,5]thieno[3,2-d]pyrimidin-4(3H)-one for 9-(dimethylamino)-3-(4-ethylphenyl)-4a,9b-dihydropyrido[3′,2′:4,5]thieno[3,2-d]pyrimidin-4(3H)-one. ¹H NMR (300 MHz, DMSO-d₆) δ 1.53-1.73 (m, 8H), 2.88 (s, 3H), 3.40 (s, br, 4H), 7.51 (d, J=5 Hz, 1H), 8.56 (s, 1H), 8.61 (d, J=5 Hz, 1H). MS (DCI/NH₃) m/z 360 (M+H)⁺.

Biological Activity

In Vitro Data—Determination of Inhibition Potencies in Rat Brain Membranes Binding.

mGluR1 binding assay was performed using rat cerebellum membrane preparation using [3H]-R214127 (9 Ci/mmol) as radioligand (Lavreysen et al., Mol Pharmacol, Vol. 63 pages 1082-93, 2003) with the exception of non-specific binding that was determined in the presence of 1 μM LY-456066 (Kingston et al. Neuroscience Abstract # 575.2, 2003). Specific binding was obtained by calculating the difference between total binding and non-specific. Radioligand saturation binding data were analyzed using Prism GraphPad software (San Diego, Calif.). Competition binding data were analyzed by non-linear regression curve fitting. Ki values were determined by the method of Cheng and Prusoff (Cheng and Prusoff, 1973).

The compounds of the present invention were found to be antagonists of the mGlu R1 receptor subtype with Ki from 1.10E-09 M to 5.20E-07M (62 compounds tested).

In Vivo Data—Determination of Antinociceptive Effect

All animal handling and experimental procedures were approved by an IACUC Committee.

Spinal Nerve Ligation: A model of spinal nerve ligation-induced neuropathic pain was produced using the procedure originally described by Kim and Chung (Kim and Chung, Pain, Vol. 50 pages 355-363, 1992). The left L5 and L6 spinal nerves of the rat were isolated adjacent to the vertebral column and tightly ligated with a 5-0 silk suture distal to the DRG, and care was taken to avoid injury of the L4 spinal nerve. Sham rats underwent the same procedure, but without nerve ligation. All animals were allowed to recover for at least 1 week and not more than 3 weeks prior to assessment of mechanical allodynia. Mechanical allodynia in the left hind paw was confirmed by comparing the paw withdrawal threshold in grams for the injured left paw and the uninjured right paw. Mechanical allodynia was measured using calibrated von Frey filaments (Stoelting, Wood Dale, Ill.). Rats were placed into inverted individual plastic containers (20×12.5×20 cm) on top of a suspended wire mesh grid, and acclimated to the test chambers for 20 min. The von Frey filaments were presented perpendicularly to the plantar surface of the selected hind paw, and then held in this position for approximately 8 sec with enough force to cause a slight bend in the filament. Positive responses included an abrupt withdrawal of the hind paw from the stimulus, or flinching behavior immediately following removal of the stimulus. A 50% withdrawal threshold was determined using an up-down procedure (Dixon, Ann. Rev. Pharmacol. Toxicol., Vol. 20 pages 441-462, 1980). Prior to compound administration, animals demonstrating motor deficit or failure to exhibit subsequent mechanical allodynia were excluded from further studies. The antinociceptive activity of a test compound was determined by comparing its ability to increase the paw withdrawal threshold of the injured left paw relative to vehicle (0%) and the uninjured right paw (100%). Activity of test compounds was determined 60 minutes after an oral dose or 30 minutes after an intraperitoneal dose. Dose-response curves as well as single dose responses were performed. Representative compounds of the present invention exhibited antinociceptive activity in this assay. The ED₅₀ for 3 compounds tested ranged from 30 μmol/kg to 55 μmol/kg (ip dosing).

Complete Freund's adjuvant-induced thermal hyperalgesia (CFA): The assay is described in Pircio et al. Eur J. Pharmacol. Vol. 31(2) pages 207-215 (1975). Chronic inflammatory hyperalgesia was induced in one group of rats following the injection of complete Freund's adjuvant (CFA, 50%, 150 μL) into the plantar surface of the right hindpaw 48 hours prior to testing. Thermal nociceptive thresholds were measured in three different groups of rats. Unilateral inflammation was induced by injecting 150 μl of a 50% solution of complete Freund's adjuvant (CFA) (Sigma Chemical Co., St. Louis, Mo.) in physiological saline into the plantar surface of the right hindpaw of the rat. The hyperalgesia to thermal stimulation was determined 48 hr after CFA injections using a commercially available paw thermal stimulator (UARDG, Department of Anesthesiology, University of California, San Diego, La Jolla, Calif.). Rats were placed individually in Plexiglass cubicles mounted on a glass surface maintained at 30° C., and allowed a 30 min habituation period. A thermal stimulus, in the form of radiant heat emitted from a focused projection bulb, was then applied to the plantar surface of each hind paw. The stimulus current was maintained at 4.5 Amp and the maximum time of exposure was set at 20 sec to limit possible tissue damage. In each test session, each rat was tested in 3 sequential trials at approximately 5 min intervals. Paw withdrawal latencies were calculated as the mean of the two shortest latencies. The antinociceptive activity of a test compound was determined by comparing its ability to increase the paw withdrawal threshold of the injured right paw relative to vehicle (0%) and the uninjured left paw (100%). Activity of test compounds was determined 60 minutes after an oral dose or 30 minutes after an intraperitoneal dose. Dose-response curves as well as single dose responses were performed. Representative compounds of the present invention exhibited antinociceptive activity in this assay. The ED₅₀ₛ were determined based on the oral administration. The ED₅₀ for 13 compounds tested ranged from 8 μmol/kg to 69 μmol/kg (ip dosing).

What is claimed is:

1. A compound of formula (I) 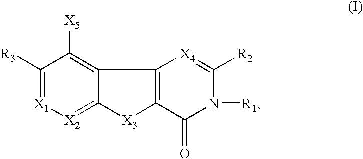
or a pharmaceutically acceptable salt or prodrug thereof, wherein
R₁ is selected from the group consisting of alkyl, aryl, cycloalkyl, heterocycle and heteroaryl;
R₂ is selected from the group consisting of hydrogen and alkyl,
R₃ is selected from the group consisting of hydrogen, alkoxyl, aryloxyl, cyano, halogen, heteroalkoxyl, and heteroaryloxyl;
X₁ is selected from the group consisting of —N—, —N⁺(O⁻)— and —C(R₄)—;
X₂ is selected from the group consisting of —N—, —N⁺(O⁻)— and —C(R₅)—;
X₃ is selected form the group consisting of S, O, and NH;
X₄ is selected from the group consisting of N and —C(R₆)—;
X₅ is selected from the group consisting of —NRₐR_{b} and —N⁺(O⁻)RₐR_{b};
R₄ and R₅ are each independently selected from the group consisting of hydrogen, alkyl, haloalkyl, hydroxy, and hydroxyalkyl;
R₆ are each independently selected from the group consisting of hydrogen, alkyl, haloalkyl, hydroxy, and hydroxyalkyl; and
Rₐ and R_{b} are each independently selected from the group consisting of hydrogen, alkyl, alkoxyalkyl, haloalkyl, hydroxyalkyl, arylalkyl, heteroarylalkyl and heterocyclealkyl, R_{c}R_{d}Nalkyl, cyanoalkyl, and cycloalkyl, wherein R_{c} and R_{d} are independently selected from the group consisting of hydrogen and alkyl;
alternatively, Rₐ and R_{b} taken together with the nitrogen to which they are attached form a heterocycle;
with the proviso that
if X₁ is N, then X₂ is —C(R₅)—, and
if X₂ is N, then X₁ is —C(R₄)—; and
the compound is not
9-Dimethylamino-3-(p-tolyl)-3H-5-thia-1,3,6-triazafluoren-4-one;
9-Dimethylamino-3-(p-methoxyphenyl)-3H-5-thia-1,3,6-triazafluoren-4-one;
9-Dimethylamino-3-(p-chlorophenyl)-3H-5-thia-1,3,6-triazafluoren-4-one; and
9-Dimethylamino-3-(p-phenyl)-3H-5-thia-1,3,6-triazafluoren-4-one.

2. The compounds according to claim 1 wherein
X₁ is —C(R₄)—;
X₂ is N—;
X₃ is S; and
X₄ is N.

3. The compound according to claim 2 wherein
X₅ is NRₐR_{b}.

4. The compound according to claim 3 wherein
Rₐ and R_{b} are each independently selected from the group consisting of hydrogen, and alkyl;
R₃ is hydrogen; and
R₄ is hydrogen.

5. The compound according to claim 4 wherein
R₁ is aryl, wherein aryl is phenyl; and
R₂ is hydrogen.

6. The compound according to claim 5 wherein said compound is selected from the group consisting of:
9-Dimethylamino-3-(o-tolyl)-3H-5-thia-1,3,6-triazafluoren-4-one;
9-Dimethylamino-3-(m-tolyl)-3H-5-thia-1,3,6-triazafluoren-4-one;
9-Dimethylamino-3-(o-hydroxyphenyl)-3H-5-thia-1,3,6-triazafluoren-4-one;
9-Dimethylamino-3-(m-fluorophenyl)-3H-5-thia-1,3,6-triazafluoren-4-one;
9-Dimethylamino-3-(p-fluorophenyl)-3H-5-thia-1,3,6-triazafluoren-4-one;
9-Dimethylamino-3-(m-chlorophenyl)-3H-5-thia-1,3,6-triazafluoren-4-one;
9-Dimethylamino-3-(p-bromophenyl)-3H-5-thia-1,3,6-triazafluoren-4-one;
9-Dimethylamino-3-(p-trifluoromethylphenyl)-3H-5-thia-1,3,6-triazafluoren-4-one;
9-Dimethylamino-3-(2,4-dimethylphenyl)-3H-5-thia-1,3,6-triazafluoren-4-one;
9-Dimethylamino-3-(2,4-dichlorophenyl)-3H-5-thia-1,3,6-triazafluoren-4-one;
9-Dimethylamino-3-(2,5-dichlorophenyl)-3H-5-thia-1,3,6-triazafluoren-4-one;
9-Dimethylamino-3-(4-ethylphenyl)-3H-5-thia-1,3,6-triazafluoren-4-one;
9-Dimethylamino-3-(3-fluoro-4-methylphenyl)-3H-5-thia-1,3,6-triazafluoren-4-one
9-Dimethylamino-3-(4-fluoro-2-methylphenyl)-3H-5-thia-1,3,6-triazafluoren-4-one
3-(3-bromophenyl)-9-(dimethylamino)pyrido[3′,2′:4,5]thieno[3,2-d]pyrimidin-4(3H)-one
3-(4-ethylphenyl)-9-(methylamino)pyrido[3′,2′:4,5]thieno[3,2-d]pyrimidin-4(3H)-one
9-Dimethylamino-3-(2′-methyl-biphenyl-3-yl)-3H-pyrido[3′,2′:4,5]thieno[3,2-d]pyrimidin-4-one
9-Dimethylamino-3-(2′-methoxy-biphenyl-3-yl)-3H-pyrido[3′,2′:4,5]thieno[3,2-d]pyrimidin-4-one
3-(2′-Chloro-biphenyl-3-yl)-9-dimethylamino-3H-pyrido[3′,2′:4,5]thieno[3,2-d]pyrimidin-4-one
9-(diethylamino)-3-(3-fluoro-4-methylphenyl)pyrido[3′,2′:4,5]thieno[3,2-d]pyrimidin-4(3H)-one
9-(N,N-Ethylmethylamino)-3-(4′-ethylphenyl)-3H-5-thia-1,3,6-triazafluoren-4-one
9-(diethylamino)-3-(4-methylphenyl)pyrido[3′,2′:4,5]thieno[3,2-d]pyrimidin-4(3H)-one
9-Dimethylamino-3-(2′-hydroxy-biphenyl-3-yl)-3H-pyrido[3′,2′:4,5]thieno[3,2-d]pyrimidin-4-one
3-(9-Dimethylamino-4-oxo-4H-pyrido[3′,2′:4,5]thieno[3,2-d]pyrimidin-3-yl)-benzonitrile
9-Dimethylamino-3-(3-thiophen-3-yl-phenyl)-3H-pyrido[3′,2′:4,5]thieno[3,2-d]pyrimidin-4-one
3-(9-Dimethylamino-4-oxo-4H-pyrido[3′,2′:4,5]thieno[3,2-d]pyrimidin-3-yl)-N-hydroxy-benzamidine
9-Dimethylamino-3-[3-(5-phenyl-[1,2,4]oxadiazol-3-yl)-phenyl]-3H-pyrido[3′,2′:4,5]thieno[3,2-d]pyrimidin-4-one
9-Dimethylamino-3-(3-{1-[(E)-methoxyimino]-ethyl}-phenyl)-3H-pyrido[3′,2′:4,5]thieno[3,2-d]pyrimidin-4-one
9-Dimethylamino-3-(3-{1-hydroxyimino-ethyl}-phenyl)-3H-pyrido[3′,2′:4,5]thieno[3,2-d]pyrimidin-4-one
3-(4-acetylphenyl)-9-(methylamino)pyrido[3′,2′:4,5]thieno[3,2-d]pyrimidin-4(3H)-one

7. The compound according to claim 3 wherein
Rₐ and R_{b} are each independently selected from the group consisting of alkyl,
alkoxyalkyl, R_{c}R_{d}Nalkyl, heteroarylalkyl, haloalkyl, cyanoalkyl and cycloalkyl;
R₁ is aryl, wherein aryl is phenyl;
R₂ is hydrogen;
R₃ is hydrogen; and
R₄ is hydrogen.

8. The compound according to claim 7 wherein said compound is selected from the group consisting of:
9-(2′-Methoxyethylmethylamino)-3-(4′-ethylphenyl)-3H-5-thia-1,3,6-triazafluoren-4-one;
9-(2′-Dimethylaminoethylmethylamino)-3-(4′-ethylphenyl)-3H-5-thia-1,3,6-triaza-fluoren-4-one;
9-[2′-(2″-Pyridinylethylmethylamino)]-3-(4′-ethylphenyl)-3H-5-thia-1,3,6-triaza-fluoren-4-one;
9-(2′,2′,2′-Trifluoroethylamino)-3-(4′-ethylphenyl)-3H-5-thia-1,3,6-triazafluoren-4-one;
9-(1′-Cyanomethylamino)-3-(4′-ethylphenyl)-3H-5-thia-1,3,6-triaza-fluoren-4-one; and
9-Cyclopropylamino-3-(4′-ethylphenyl)-3H-5-thia-1,3,6-triazafluoren-4-one.

9. The compound according to claim 4 wherein
R₁ is aryl, wherein aryl is phenyl; and
R₂ is alkyl.

10. The compound according to claim 9 wherein said compound is selected from the group consisting of:
9-(dimethylamino)-3-(4-ethylphenyl)-2-methylpyrido[3′,2′:4,5]thieno[3,2-d]pyrimidin-4(3H)-one;
9-(dimethylamino)-2-methyl-3-(4-methylphenyl)pyrido[3′,2′:4,5]thieno[3,2-d]pyrimidin-4(3H)-one; and
2-butyl-9-(dimethylamino)-3-(4-ethylphenyl)pyrido[3′,2′:4,5]thieno[3,2-d]pyrimidin-4(3H)-one.

11. The compound according to claim 4 wherein
R₁ is heteroaryl; and
R₂ is hydrogen.

12. The compound according to claim 11 wherein said compound is selected from the group consisting of:
9-Dimethylamino-3-(3,4-methylenedioxyphenyl)-3H-5-thia-1,3,6-triazafluoren-4-one;
9-Dimethylamino-3-(thiozol-2-yl)-3H-5-thia-1,3,6-triazafluoren-4-one; and
9-Dimethylamino-3-(2′-methoxy-5′-pyridinyl)-3H-5-thia-1,3,6-triazafluoren-4-one.

13. The compound according to claim 4 wherein
R₁ is alkyl; and
R₂ is hydrogen.

14. The compound according to claim 13 wherein said compound is
9-Dimethylamino-3-(2′,2′-dimethyl-1′-propyl)-3H-5-thia-1,3,6-triazafluoren-4-one.

15. The compound according to claim 4 wherein
R₁ is cycloalkyl; and
R₂ is hydrogen.

16. The compound according to claim 15 wherein said compound is selected from the group consisting of:
9-(dimethylamino)-3-(4-methylcyclohexyl)pyrido[3′,2′:4,5]furo[3,2-d]pyrimidin-4(3H)-one; and
3-cycloheptyl-9-(diethylamino)pyrido[3′,2′:4,5]thieno[3,2-d]pyrimidin-4(3H)-one.

17. The compound according to claim 4 wherein
R₁ is heterocycle; and
R₂ is hydrogen.

18. The compound according to claim 17 wherein said compound is
9-Dimethylamino-3-(N-hexamethyleneiminyl)-3H-5-thia-1,3,6-triazafluoren-4-one.

19. The compound according to claim 3 wherein
Rₐ and R_{b} taken together with the nitrogen to which they are attached form a heterocycle;
R₁ is aryl, wherein aryl is phenyl;
R₂ is hydrogen;
R₃ is hydrogen; and
R₄ is hydrogen.

20. The compound according to claim 19 wherein said compound is selected from the group consisting of:
3-(3-fluoro-4-methylphenyl)-9-pyrrolidin-1-ylpyrido[3′,2′:4,5]thieno[3,2-d]pyrimidin-4(3H)-one;
3-(4-methylphenyl)-9-pyrrolidin-1-ylpyrido[3′,2′:4,5]thieno[3,2-d]pyrimidin-4(3H)-one; and
9-N-Azetidinyl-3-(4′-ethylphenyl)-3H-5-thia-1,3,6-triazafluoren-4-one

21. The compound according to claim 3 wherein
Rₐ and R_{b} are hydrogen;
R₁ is aryl, wherein aryl is phenyl;
R₂ is hydrogen;
R₃ is halogen; and
R₄ is hydrogen.

22. The compound according to claim 21 wherein said compound is
8-Chloro-9-dimethylamino-3-(4-ethylphenyl)-3H-5-thia-1,3,6-triazafluoren-4-one

23. The compound according to claim 2 wherein
X₅ is —N⁺(O⁻)RₐR_{b};
Rₐ and R_{b} are each independently selected from the group consisting of hydrogen, and alkyl;
R₁ is selected from the group consisting of aryl and heterocycle; and
R₂ is hydrogen.

24. The compound according to claim 23 wherein R₁ is aryl.

25. The compound according to claim 24 wherein said compound is
9-(dimethylnitroryl)-3-(4-ethylphenyl)-4a,9b-dihydropyrido[3′,2′:4,5]thieno[3,2-d]pyrimidin-4(3H)-one.

26. The compound according to claim 23 wherein
R₁ is heterocycle.

27. The compound according to claim 26 wherein said compound is
3-azepan-1-yl-9-(dimethylnitroryl)-4a,9b-dihydropyrido[3′,2′:4,5]thieno[3,2-d]pyrimidin-4(3H)-one

28. The compounds according to claim 1 wherein
X₁ is —C(R₄)—;
X₂ is —C(R₅)—;
X₃ is S; and
X₄ is N.

29. The compounds according to claim 28 wherein
X₅ is NRₐR_{b};
R₁ is aryl, wherein aryl is phenyl; and
R₂ is hydrogen.

30. The compound according to claim 29 wherein said compound is
9-(dimethylamino)-3-(4-ethylphenyl)[1]benzothieno[3,2-d]pyrimidin-4(3H)-one

31. The compounds according to claim 1 wherein
X₁ is N—;
X₂ is —C(R₅)—;
X₃ is S; and
X₄ is N.

32. The compounds according to claim 31 wherein
X₅ is NRₐR_{b};
R₁ is aryl, wherein aryl is phenyl; and
R₂ is hydrogen.

33. The compound according to claim 32 wherein
Rₐ and R_{b} are each independently selected from the group consisting of alkyl and hydrogen

34. The compound according to claim 33 wherein said compound is selected from the group consisting of:
9-(dimethylamino)-3-(4-ethylphenyl)pyrido[4′,3′:4,5]thieno[3,2-d]pyrimidin-4(3H)-one;
9-(dimethylamino)-3-(3-fluoro-4-methylphenyl)pyrido[4′,3′:4,5]thieno[3,2-d]pyrimidin-4(3H)-one; and
9-(dimethylamino)-3-(4-methylphenyl)pyrido[4′,3′:4,5]thieno[3,2-d]pyrimidin-4(3H)-one.

35. The compound according to claim 34 wherein
Rₐ and R_{b} taken together with the nitrogen to which they are attached form a heterocycle.

36. The compound according to claim 35 wherein said compound is selected from the group consisting of:
3-(4-ethylphenyl)-9-pyrrolidin-1-ylpyrido[4′,3′:4,5]thieno[3,2-d]pyrimidin-4(3H)-one;
3-(3-fluoro-4-methylphenyl)-9-pyrrolidin-1-ylpyrido[4′,3′:4,5]thieno[3,2-d]pyrimidin-4(3H)-one; and
3-(4-methylphenyl)-9-pyrrolidin-1-ylpyrido[4′,3′:4,5]thieno[3,2-d]pyrimidin-4(3H)-one.

37. The compound according to claim 31 wherein
X₅ is NRₐR_{b};
R₁ is cycloalkyl; and
R₂ and R₅ are hydrogen.

38. The compound according to claim 37 wherein
Rₐ and R_{b} are each independently selected from the group consisting of alkyl and hydrogen

39. The compound according to claim 38 wherein said compound is
3-cycloheptyl-9-(dimethylamino)pyrido[4′,3′:4,5]thieno[3,2-d]pyrimidin-4(3H)-one.

40. The compound according to claim 37 wherein
Rₐ and R_{b} taken together with the nitrogen to which they are attached form a heterocycle.

41. The compound according to claim 40 wherein said compound is
3-cycloheptyl-9-pyrrolidin-1-ylpyrido[4′,3′:4,5]thieno[3,2-d]pyrimidin-4(3H)-one.

42. The compounds according to claim 1 wherein
X₁ is —C(R₄)—;
X₂ is —N⁺(O⁻)—
X₃ is S; and
X₄ is N.

43. The compounds according to claim 42 wherein
X₅ is NRₐR_{b}.
Rₐ and R_{b} are each independently selected from the group consisting of hydrogen and alkyl;
R₃ is hydrogen; and
R₄ is hydrogen.

44. The compound according to claim 43 wherein
R₁ is aryl, wherein aryl is phenyl; and
R₂ is hydrogen.

45. The compound according to claim 40 wherein said compound is
9-(dimethylamino)-3-(4-ethylphenyl)pyrido[3′,2′:4,5]thieno[3,2-d]pyrimidin-4(3H)-one 6-oxide.

46. A pharmaceutical composition comprising a therapeutically effective amount of a compound of formula (I) according to claim 1 in combination with a pharmaceutically acceptable carrier.

47. A method of treating a disorder wherein the disorder is ameliorated by inhibiting metabotropic glutamate (mGlu) receptor, wherein said disorder is selected form the group consisting of pain, neurodegeneration, Parkinson's disease, addiction to psychostimulant drugs, anxiety, depression, and convulsive states, in a host mammal in need of such treatment comprising administering a therapeutically effective amount of a compound of formula (I) according to claim 1, or a pharmaceutically acceptable salt thereof.

48. The method according to claim 47, wherein said disorder is pain.

49. The method according to claim 47, wherein said disorder is neurodegeneration.

50. The method according to claim 47, wherein said disorder is Parkinson's disease.

51. The method according to claim 47, wherein said disorder involves convulsive states.

52. The method according to claim 47, wherein said disorder is anxiety.

53. The method according to claim 47, wherein said disorder is depression.

54. The method according to claim 47, wherein said disorder involves addiction to psychostimulant drugs.
